SEQUENCE LISTING (61 sequences)

SEQ ID NO: 1 - protein, 23 aa, Artificial sequence
  Designed peptide
  PEPTIDE (1)..(23): GFP
TETKTVTITRPKMTWTFTHTVTG

SEQ ID NO: 2 - protein, 23 aa, Artificial sequence
  Designed peptide
  misc_feature (1)..(1): x=L-propargylglycin
  misc_feature (22)..(22): x=L-4-azido-2-aminobutanoic acid
  misc_feature (23)..(23): G-OH
XETKTVTITRPKMTWTFTHTVXG

SEQ ID NO: 3 - protein, 21 aa, Artificial sequence
  Designed peptide
  PEPTIDE (1)..(21): GFP
KTGTQNLTGPGRTHTQTATEG

SEQ ID NO: 4 - protein, 6 aa, Artificial sequence
  Designed peptide
  PEPTIDE (1)..(6): Peptide
HRMVRG

SEQ ID NO: 5 - protein, 20 aa, Artificial sequence
  Designed peptide
  PEPTIDE (1)..(20): Peptide
ETDTYTETYPGYTSTWTITD

SEQ ID NO: 6 - protein, 18 aa, Artificial sequence
  Designed peptide
  PEPTIDE (1)..(18): Peptide
TWTDTATEPGYTMTATGT

SEQ ID NO: 7 - protein, 18 aa, Artificial sequence
  Designed peptide
  PEPTIDE (1)..(18): Peptide
TKTDRVTEPGRTMTFTGT

SEQ ID NO: 8 - protein, 21 aa, Artificial sequence
  Designed peptide
  PEPTIDE (1)..(21): X=L-propargylglycin
XETDTYTETYPGYTSTWTITD

SEQ ID NO: 9 - protein, 21 aa, Artificial sequence
  Designed peptide
  PEPTIDE (1)..(21): X1=L-propargylglycin; X19=L-4-azido-2- aminobutanoic acid
EXDTYTETYPGRTITWTIXDG

SEQ ID NO: 10 - protein, 21 aa, Artificial sequence
  Designed peptide
  PEPTIDE (1)..(21): X2= L-4-azido-2-aminobutanoic acid ; X20=L- propargylglycin
GEXITSTVTDPGKTDTVQNXG

SEQ ID NO: 11 - protein, 22 aa, Artificial sequence
  Designed peptide
  PEPTIDE (1)..(22): Peptide
TWTETYTWTEPGDTQTLTITNT

SEQ ID NO: 12 - protein, 18 aa, Artificial sequence
  Designed peptide
  PEPTIDE (1)..(18): Peptide
TWTKTGTAPGLTVRYTYT

SEQ ID NO: 13 - protein, 18 aa, Artificial sequence
  Designed peptide
  PEPTIDE (1)..(18): Peptide
ETYTETYPGYTSTWTIDD

SEQ ID NO: 14 - protein, 18 aa, Artificial sequence
  Designed peptide
  PEPTIDE (1)..(18): Peptide
TRTLTYTEPGITQTKTEA

SEQ ID NO: 15 - protein, 20 aa, Artificial sequence
  Designed peptide
  PEPTIDE (1)..(20): Peptide
NTVTNTMTRPGVTETVTQTD

SEQ ID NO: 16 - protein, 18 aa, Artificial sequence
  Designed peptide
  PEPTIDE (1)..(18): Peptide
TMTDTDTYPGFTDTLTHA

SEQ ID NO: 17 - protein, 20 aa, Artificial sequence
  Designed peptide
  PEPTIDE (1)..(20): Peptide
HTWTDTLTRPGYTVTHTLTL

SEQ ID NO: 18 - protein, 20 aa, Artificial sequence
  Designed peptide
  PEPTIDE (1)..(20): X2=L-propargylglycin  X19=L-4-azido-2- aminobutanoic acid
GXSTWTMTNPGWTKTHTLXG

SEQ ID NO: 19 - protein, 23 aa, Artificial sequence
  Designed peptide
  PEPTIDE (1)..(23): X3= L-4-azido-2-aminobutanoic acid  ; X21=L- propargylglycin
GGHXWTDTLTRPGYTVTHTLXLG

SEQ ID NO: 20 - protein, 20 aa, Artificial sequence
  Designed peptide
  PEPTIDE (1)..(20): Peptide
ATNTLTMTWPGRTNTDTFTW

SEQ ID NO: 21 - protein, 18 aa, Artificial sequence
  Designed peptide
  PEPTIDE (1)..(18): Peptide
TKTRTYTIPGERYTDTWA

SEQ ID NO: 22 - protein, 19 aa, Artificial sequence
  Designed peptide
  PEPTIDE (1)..(19): Peptide
TLTFTATRPGLTKTITITL

SEQ ID NO: 23 - protein, 19 aa, Artificial sequence
  Designed peptide
  PEPTIDE (1)..(19): Peptide
DTVTKTFTWPGAKLTFTKT

SEQ ID NO: 24 - protein, 20 aa, Artificial sequence
  Designed peptide
  PEPTIDE (1)..(20): Peptide
KTWTLTHTKPGNTATDTHTI

SEQ ID NO: 25 - protein, 18 aa, Artificial sequence
  Designed peptide
  PEPTIDE (1)..(18): Peptide
RLTWTMTIPGLTLTLTDT

SEQ ID NO: 26 - protein, 18 aa, Artificial sequence
  Designed peptide
  PEPTIDE (1)..(18): Peptide
TWTLTWTKPGQEQTMTHA

SEQ ID NO: 27 - protein, 20 aa, Artificial sequence
  Designed peptide
  PEPTIDE (1)..(20): Peptide
YTLTDTETYPGHTRTATQTE

SEQ ID NO: 28 - protein, 17 aa, Artificial sequence
  Designed peptide
  PEPTIDE (1)..(17): Peptide
WEHTHTETSPGNTQTST

SEQ ID NO: 29 - protein, 17 aa, Artificial sequence
  Designed peptide
  PEPTIDE (1)..(17): Peptide
WEHTHTETSPGNTYTST

SEQ ID NO: 30 - protein, 24 aa, Artificial sequence
  Designed peptide
  PEPTIDE (1)..(24): X2=L-4-azido-2-aminobutanoic acid   ; X23=L- propargylglycin
EXFTMEQTWTGPGSTKTFTFTHXG

SEQ ID NO: 31 - protein, 29 aa, Artificial sequence
  Designed peptide
  PEPTIDE (1)..(29): Peptide
LTFTFTVTPGTFTWTGTKPGETYTFTRTE

SEQ ID NO: 32 - protein, 23 aa, Artificial sequence
  Designed peptide
  PEPTIDE (1)..(23): X1= L-propargylglycin ; X22= L-4-azido-2- aminobutanoic acid  ; G23=G-OH
XKTKTWTMTGPGGEKTRTLTAXG

SEQ ID NO: 33 - protein, 24 aa, Artificial sequence
  Designed peptide
  PEPTIDE (1)..(24): X1=L-propargylglycin  ; X23=L-4-azido-2- aminobutanoic acid
XWTNTGTYTIPGVTVTMTETVAXE

SEQ ID NO: 34 - protein, 18 aa, Artificial sequence
  Designed peptide
  PEPTIDE (1)..(18): X1= L-propargylglycin ; X18=L-4-azido-2- aminobutanoic acid
XTVTGTLYPGTLLGFETX

SEQ ID NO: 35 - protein, 24 aa, Artificial sequence
  Designed peptide
  PEPTIDE (1)..(24): V1= Ac-V (ACYLATED)
VTWTDTLTFTLPGVTWTITMTITE

SEQ ID NO: 36 - protein, 24 aa, Artificial sequence
  Designed peptide
  PEPTIDE (1)..(24): Peptide
HTLTKTITQTWPGKTYTITWTFTW

SEQ ID NO: 37 - protein, 18 aa, Artificial sequence
  Designed peptide
  PEPTIDE (1)..(18): X4=L-propargylglycin  ; X15=L-4-azido-2- aminobutanoic acid
KTWXLTITPGTMEIXDTV

SEQ ID NO: 38 - protein, 19 aa, Artificial sequence
  Designed peptide
  PEPTIDE (1)..(19): X1= L-propargylglycin ; X19=L-4-azido-2- aminobutanoic acid , hydroxylated
XYTYTDTTPGVTRTLTWGX

SEQ ID NO: 39 - protein, 18 aa, Artificial sequence
  Designed peptide
  PEPTIDE (1)..(18): X1=L-propargylglycin  ; X18=L-4-azido-2- aminobutanoic acid , hydroxylated
XTWTLTHTPGTMEITETX

SEQ ID NO: 40 - protein, 18 aa, Artificial sequence
  Designed peptide
  PEPTIDE (1)..(18): X3=6-NH2; X18=L-4-azido-2-aminobutanoic acid , hydroxylated
TWXTLTHTPGTMEITETX

SEQ ID NO: 41 - protein, 18 aa, Artificial sequence
  Designed peptide
  PEPTIDE (1)..(18): Peptide
TTVTGTLYPGTLLGFETT

SEQ ID NO: 42 - protein, 18 aa, Artificial sequence
  Designed peptide
  PEPTIDE (1)..(18): Peptide
CTVTGTLYPGTLLGFETC

SEQ ID NO: 43 - protein, 18 aa, Artificial sequence
  Designed peptide
  PEPTIDE (1)..(18): Peptide
TTVTGTLYPGTLLGAATT

SEQ ID NO: 44 - protein, 18 aa, Artificial sequence
  Designed peptide
  PEPTIDE (1)..(18): X1= L-4-azido-2-aminobutanoic acid  ; X18= L- propargylglycin
XTVTGTLYPGTLLGFETX

SEQ ID NO: 45 - protein, 23 aa, Artificial sequence
  Designed peptide
  PEPTIDE (1)..(23): Peptide
TITKTARYTMPGKTLTKTGTLTG

SEQ ID NO: 46 - protein, 23 aa, Artificial sequence
  Designed peptide
  PEPTIDE (1)..(23): X1= L-propargylglycin ; X22=L-4-azido-2- aminobutanoic acid
XITKTARYTMPGKTLTKTGTLXG

SEQ ID NO: 47 - protein, 25 aa, Artificial sequence
  Designed peptide
  PEPTIDE (1)..(25): Residues 1-12 D-amino acid residues; Residues 14-24 D-amino acid residues; G25=G-OH
TLTMTWTYTHTPGTITWTYTDTTTG

SEQ ID NO: 48 - protein, 25 aa, Artificial sequence
  Designed peptide
  PEPTIDE (1)..(25): Residues 1-12 D-amino acid residues; Residues 14-24 D-amino acid residues; G25=G-OH; X1= D-propargylglycin ; X24= D-3-azido-2-aminobutanoic acid; G25=G-OH
XLTMTWTYTHTPGTITWTYTDTTXG

SEQ ID NO: 49 - protein, 23 aa, Artificial sequence
  Designed peptide
  PEPTIDE (1)..(23): X4= D-3-azido-2-aminobutanoic acid ; X21= D-propargylglycin
IHVXVTTRTMPGHPIAGADAXTG

SEQ ID NO: 50 - protein, 25 aa, Artificial sequence
  Designed peptide
  PEPTIDE (1)..(25): All D-amino acid residues (except for the glycine residues)
TQTMTGTLTWTPGTQTNTLTWTFTG

SEQ ID NO: 51 - protein, 25 aa, Artificial sequence
  Designed peptide
  PEPTIDE (1)..(25): All D-amino acid residues (except for the glycin residues). X1= D-4-azido-2-aminobutanoic acid  ; X24= D- propargylglycin
XQTMTGTLTWTPGTQTNTLTWTFXG

SEQ ID NO: 52 - protein, 15 aa, Artificial sequence
  Designed peptide
  PEPTIDE (1)..(15): All D-amino acid residues (except the glycine residues). X1=D-4-azido-2-aminobutanoic acid   ; X14= D- propargylglycin
XWTFTLPQGTMTNXG

SEQ ID NO: 53 - protein, 28 aa, Artificial sequence
  Designed peptide
  PEPTIDE (1)..(28): All D-amino acid residues (except the glycine residues). F28=F-OH
ETLTWTGTVTVTFPGITMTTTETMTFTF

SEQ ID NO: 54 - protein, 18 aa, Artificial sequence
  Designed peptide
  PEPTIDE (1)..(18): All D-amino acid residues (except the glycine residues). X1= D-4-azido-2-aminobutanoic acid  ; X18= D- propargylglycin, hydroxylated
XWTVTVTFPGITMTTTFX

SEQ ID NO: 55 - protein, 21 aa, Artificial sequence
  Designed peptide
  PEPTIDE (1)..(21): Peptide
KTQTYNGTGPGRTGTVTYTEG

SEQ ID NO: 56 - protein, 21 aa, Artificial sequence
  Designed peptide
  PEPTIDE (1)..(21): Peptide
KTYTYNYTGPGRTSTATLTEG

SEQ ID NO: 57 - protein, 14 aa, Artificial sequence
  Designed peptide
  PEPTIDE (1)..(14): Peptide
TYTYTYPGLTRTHT

SEQ ID NO: 58 - protein, 12 aa, Artificial sequence
  Designed peptide
  PEPTIDE (1)..(12): Peptide
TTYTYPGDTFTI

SEQ ID NO: 59 - protein, 14 aa, Artificial sequence
  Designed peptide
  PEPTIDE (1)..(14): Peptide
TFTFTFPGLTRTHT

SEQ ID NO: 60 - protein, 22 aa, Artificial sequence
  Designed peptide
  PEPTIDE (1)..(22): Peptide
TDTRTYTYTVPGRTRTRTWTET

SEQ ID NO: 61 - protein, 21 aa, Artificial sequence
  Designed peptide
  PEPTIDE (1)..(21): Peptide
DTITYTYTGPGRTDTETNTEG

(19) United States
(12) Patent Application Publication
(10) Pub. No.: US 2019/0284243 A1
(43) Pub. Date: Sep. 19, 2019

(54) BINDING PEPTIDES

(71) Applicant: University of Copenhagen, Copenhagen K (DK)

(72) Inventors: Morten MELDAL, Copenhagen NV (DK); Hongxia HU, Copenhagen N (DK); Ming LI, Kgs. Lyngby (DK); Niklas Henrik FISCHER, Rødovre (DK); Sanne SCHOFFELEN, Copenhagen NV (DK); Frederik DINESS, Ølstykke (DK)

(21) Appl. No.: 16/349,007

(22) PCT Filed: Nov. 9, 2017
(86) PCT No.: PCT/EP2017/078766

§ 371 Date: May 10, 2019

(30) Foreign Application Priority Data
Nov. 11, 2016 (DK) PA 2016 70899

(51) Int. Cl. C07K 14/00; G16B 15/30; G01N 33/68

(52) U.S. Cl. CPC C07K 14/001; G01N 33/6857; G16B 15/30

(57) ABSTRACT

The present invention relates to peptides binding target compounds including other peptides with high specificity and affinity.

### TECHNICAL FIELD

The present invention relates to the field of peptides binding target compounds, including other peptides with high specificity and affinity as well as peptides comprising non-naturally occurring amino acids.

### BACKGROUND

Antibodies are being applied in a variety of methods involving biomolecular recognition. They are highly specific and are used in diagnosis and as therapeutic agents. Antibodies may be generated by immunization with antigens and the immune system show preference for generation of antibodies to certain sites on the antigen, the so-called epitopes. Binding constants for antibodies are in the range of 10⁻¹⁰-10⁻⁵ M and is centred around 10⁻⁷ M. The disadvantage of antibodies is that they are obtained as a polyclonal response to the antigen and lengthy processes of generating monoclonal antibodies are usually required. The expression of proteins is cumbersome and often subject to years of optimization for large scale production. For therapeutic antibodies these are often themselves immunogens and therefore furthermore needs to be “humanized” including the appropriate glycosylation, frequently only obtained in very specific human or mammalian cell lines, before they can be employed in treatment.

### SUMMARY

The present invention provides a new class of peptides, which can bind target compounds high specificity and affinity. Herein this class of peptides is referred to as β-bodies.

β-Bodies has several great advantages over conventional antibodies. Thus, β-bodies can be generated, which bind to any protein surface for which the structure is known. This means that a large variety of β-bodies can be synthetically obtained, that selectively recognize the same protein in different manners. The binding surface area of optimized β-bodies readily covers the same surface area as an antibody and the binding constants measured are of the same order as that for antibodies. Furthermore a β-body can be directly designed to interact with specific location of interest on a protein, e. g. an active site of an enzyme, a site for protein-protein interaction or a binding pocket. Accordingly, β-bodies are useful as a replacement of neutralizing antibodies. Since the β-bodies of the invention typically are small, it is unlikely that they will be generally immunogenic and in contrast to antibodies they can be used directly as therapeutic agents

β-hairpins have been described. For example US2012321697, US2012309934 and WO10047515 describe a bipodal peptide comprising a beta-hairpin region and a separate region for target binding. The target binding region has random structure and is not a part of the β-hairpin.

The present invention provides peptides, which can bind either itself or a target compound with high specificity and affinity. The peptides generally comprise a β-hairpin and/or a β-sheet that typically is designed by use of the following principles:
the β-hairpin or β-sheet has one surface (surface 1) made up by amino acid side chains, which contribute to the β-hairpin or β-sheet structure;
the β-hairpin or β-sheet has a second surface (surface 2) made up by amino acid side chains that specifically interact with the target compound.

Typically, the amino acids making up surface 1 and the amino acids making up surface 2 are positioned in alternating positions within the β-strands making up the β-hairpin or β-sheet.

Thus, the peptides of the invention comprise a very stable 3 dimensional structure in the form of a β-hairpin or β-sheet. The amino acids making up surface 2 can be designed to bind any useful target compound either by use of computer aided modelling or by screening libraries of peptides.

The invention is defined in the claims attached hereto. For example, the invention provides compounds, wherein designated “β-bodies”, wherein the β-body is a compound comprising or consisting of at least two β-strand peptide sequences connected by β-turn peptide sequence(s), wherein said β-strand peptide sequences are organized in an anti-parallel arrangement of alternating forward and reverse β-strand peptide sequences, wherein

each forward β-strand peptide sequence individually has the following sequence

Xᵣ(ZX)ₘ

and each reverse β-strand peptide sequence individually has the following sequence

(XZ)ₙXᵣ
wherein

each Z individually is Thr, a polar β-branched amino acid, non-proteinogenic α-branched amino acids that promote β-strand structure or a strand bridging amino acid, with the exception that at the most two Z in each β-strand sequence may be an amino acid, which is not one of the aforementioned; each X individually is any amino acid, β-amino acid or γ-amino acid; and each m and n individually are integers in the range of 3 to 12; and each r is an integer in the range of 0 to 5; and

and each β-turn peptide sequence individually has the following sequence

X_{q1}BUX_{q2}

wherein

each X individually is any amino acid;

each U individually is an amino acid of the formula

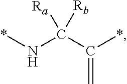

wherein Ra and Rb individually are selected from the group consisting of —H and C₁₋₆-alkyl, wherein Ra and Rb may be linked to form a cyclic structure;

B is selected from the group consisting of Pro, substituted Pro and pipecolic acid; and

each q individually is an integer in the range of 0 to 5, wherein q1-q2 is −4, −2, 0, 2 or 4.

The invention further provides
methods of identifying β-bodies according to the invention,
methods for detecting the presence of a target compound using the β-bodies of the invention,
methods for diagnosing a clinical condition using the β-bodies of the invention,
β-bodies for use in a method of treating a clinical condition
homo- and heterodimers of β-bodies
compounds comprising β-bodies covalently linked to a conjugated moiety.

### DESCRIPTION OF DRAWINGS

FIG. 1. Panel A shows a model of IL-2 bound to the two β-bodies, ligand 3 and ligand 4. Panel B, C and D shows PEGA beads linked to ligand 3 incubated with ligand 4 (Panel B)(no signal) or ligand 4 and IL2 (Panels C and D)(positive signal).

FIG. 2. Panel A shows a model of an EGFP fusion protein containing: histag—EGFP—Spacer—KTGTQNLTGPGRTHTQTATEG (SEQ ID NO: 3) bound to the hexapeptide HRMVRG immobilised on a PEGA resin bead. Panels B and C shows PEGA-beads linked to HRMVRG in the presence of the EGFP fusion protein (panel B) or in the presence of EGFP (Panel C). Panel D shows an SDS-PAGE analysis of samples obtained during purification of the aforementioned EGFP fusion protein prepared as described in Example 5. 1) cell lysate of cells expressing the EGFP fusion protein, 2) flow-through-solution, 3) wash with Milli-Q water, 4) eluate with PBS buffer, 5) protein standard, 6) cell lysate of cells expressing EGFP, 7) flow through solution. The expected sizes of the EGFP fusion protein is indicated as “GFP-Hairpin” and of EGFP as “GFP”.

FIG. 3. Panel A shows a model of IL-1 bound to the two β-bodies, ligand 1 and ligand 2. Panel B and C show PEGA beads linked to ligand 1 incubated with ligand 2 (Panel B)(no signal) or ligand 2 and IL1 (Panel C)(positive signal). Ligands 1 and 2 were mixed and incubated for 4 h (image at t=0). IL-1 was added (50 nM) and incubated for 20 min. (image at t=20).

FIG. 4. Panel A shows a model of IL-6 bound to the two β-bodies, ligand 5 and ligand 6. Panel B, C and D shows PEGA beads linked to ligand 5 incubated with ligand 6 (Panels B and C)(no signal) or ligand 6 and IL6 (Panel D)(positive signal). Ligand 5 and 6 were mixed and incubated for 4 h (image at t=0). II6 was added (20 nM) and incubated for 20 min (image at t=20). The beads were washed with pbs buffer before imaging.

FIG. 5. Schematic representation of a β-body comprising two β-sheets and/or β-hairpins connected to each other via a β-turn.

Each β-sheet and/or β-hairpin is characterized by:
one surface (surface 1) made up by amino acid side chains, which contribute to the β-hairpin or β-sheet structure (Structural site);
a second surface (surface 2) made up by amino acid side chains that specifically interact with the target compound (recognition site).

In the represented β-body, the amino acids making up surface 1 and the amino acids making up surface 2 are positioned in alternating positions within the β-strands making up the β-hairpin or β-sheet. Panel A shows a β-body with the recognition residue in an outward orientation; panel B A shows a β-body with the recognition residue in an inward orientation.

FIG. 6. β-body binding GFP in lysate from E. coli (A) untreated and (B) diluted.

FIG. 7. (A-1) Bright field image of two beads, one with a covalently linked β-body for eGFP and the other with a covalently linked β-body for interleukin 1 (IL1) both incubated with eGFP molecules. (A-2) The same image but recorded under a fluorescence microscope which shows that the β-body binds selectively to eGFP. (B-1) Bright field image of two beads, one with a covalently linked β-body for eGFP and another with a covalently linked β-body for IL1, and both are incubated with with IL1 molecules. (B-2) The same image but recorded under a fluorescence microscope which shows that the β-body binds selectively to IL1 .

FIG. 8. Beads modified with NHAc are incubated with the β-body 1-F* (A) or 2-F* (B). Beads modified with the β-body 1 are incubated with the β-body 1-F* (C) or 2-F*(D). Beads modified with the β-body 2 are incubated with the β-body 1-F* (E) or 2-F*(F).

Definitions

The term “alkyl” refers to a substituent derived from an alkane by removal of one —H.

The term “amino acid” as used herein a-amino acids, β-amino acids and γ-amino acids. Preferably, an amino acid is a compound of the following general structure I:

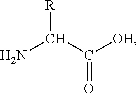

wherein R indicates the amino acid side chain. R may be —H in which case the amino acid is glycine. Apart from glycine, amino acids have the general structure II:

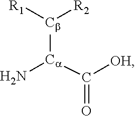

wherein R₁ and R₂ may be —H or a substituent. The α-carbon and the β-carbon atom of an amino acid is indicated as C_{α}, and C_{β}, respectively. Amino acids may be bound to each other by peptide bonds to form polypeptides of the following general structure III:

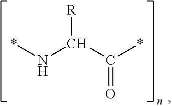

wherein n is an integer and * indicates the point of attachment to the next amino acid residue. Amino acids may be standard amino acids, but also includes other amino acids of aforementioned general structure. Amino acids may be D-stereo-isomers (referred to as D-amino acids herein) or may be L-stereo-isomers (referred to as L-amino acids herein). The amino acid may also be a cyclic amino acid such as proline, pipecolic acid or derivatives thereof.

The term “amino acid residue” refers to an amino acid monomer within a polypeptide.

An amino acid residue preferably has the general structure IV:

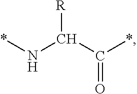

where R indicates the amino acid side chain. When the amino acid residue is not Gly, then the amino acid residue has the general structure V:

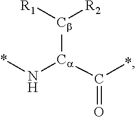

where * indicates the point of attachment to the neighbouring amino acid residue. In the most N-terminal amino residue the position indicated by * linked to N is —H, whereas in the most C-terminal amino acid residue the position indicated by * linked to C═O is —OH or —NH₂.

The term “aryl” as used herein refers to a substituent derived from an arene by removal of one —H from a C in the ring. Examples of useful aryls to be used with the present invention comprise phenyl, napthyl, anthracenyl, phenanthrenyl, pyrenyl or substituted versions thereof including substituents such as —F, —Cl, —Br, —I, —-OH, —OMe, NH₂, —CF₃, —COOH, —OPO₃H₂, or —CH₂—PO₃H₂.

The term “β-branched amino acid” refers to an amino acid wherein the β-carbon atom is branched. Thus, in β-branched amino acids, the β-carbon atom is directly covalently bound to the α-carbon and to at least 2 additional atoms, which are not —H.

The term “β-amino acid” as used herein refers to an amino acid, which has the amino group bonded to the β carbon rather than to the a carbon as in the standard amino acids.

The term “detectable label” as used herein refers to any label, which can be detected. The detectable label may for example be selected from the group consisting of radiolabels, biotin, fluorescent labels, luminescent labels and coloured labels.

The term “γ-amino acid” as used herein refers to an amino acid, which has the amino group bonded to the γ-carbon rather than to the a carbon as in the standard amino acids.

The term “K_{d}” as used herein refers to the dissociation constant. Accordingly, K_{d} may be used as a measure of the binding affinity between a β-body and its target compound. The Kd may be calculated using the following equation:

$$Kd = \frac{[ A ] [ B ]}{[ AB ]}$$

wherein [A] indicates the concentration of target compound, [B] indicates the concentration of free β-body and [AB] indicates the concentration of complex at equilibrium.

The term “inward β-body” as used herein refers to a β-body wherein the Z amino acid residue as defined in the section below “Amino acid Z”, for example a threonine, immediately precedes and follows a β-type2-turn.

The term “outward β-body” as used herein refers to a β-body wherein the recognition residues, such as the X amino acid residue as defined in the section below “Amino acid X”, immediately precede and follows a β-type2-turn.

The term “polypeptide” as used herein refers to a sequence of amino acid residues linked by peptide bonds. In general a polypeptide comprises at least 4 amino acid residues.

The term “standard amino acid” refers to the 20 amino acids encoded by the standard genetic code. The amino acids are referred to herein using standard IUPAC nomenclature. Standard amino acids are all L-amino acids.

The term “strand bridging amino acids” as used herein refers to two amino acids located on opposite strands, which are capable of forming a covalent chemical bond or a hydrogen bond across the two strands without perturbation of the strand arrangement. Covalent strand bridging include disulphide bonds and triazoles formed by Copper-Catalyzed Azide-Alkyne Cycloaddition (CuAAC)—click reactions.

β-body

The present invention relates to compounds comprising or consisting of at least two β-strand peptide sequences connected by β-turn peptide sequence(s), wherein said β-strand peptide sequences may be organized in an anti-parallel arrangement of alternating forward and reverse β-strand peptide sequences. Such compounds may also be referred to as “beta-bodies” or “β-bodies” herein.

Each forward β-strand peptide sequence may individually have the following general sequence I:

Xᵣ(ZX)ₘ

and may be any of the peptide sequences described herein below in more detail in the section “Forward β-strand peptide sequences”. In embodiments of the invention wherein the β-body comprise more than one forward β-strand sequence it is understood that even though the forward β-strand peptide sequences have the same general sequence, then each forward β-strand within a β-body may have different specific sequences.

Each reverse β-strand peptide sequence may individually have the following general sequence II:

(XZ)ₙXᵣ

and may be any of the peptide sequences described herein below in more detail in the section “Reverse β-strand peptide sequences”. In embodiments of the invention wherein the β-body comprise more than one reverse β-strand sequence it is understood that even though the reverse β-strand peptide sequences have the same general sequence, then each reverse β-strand within a β-body may have different specific sequences.

The amino acid denoted Z may be any of the amino acids described below in the section “Amino acid Z”, whereas the amino acid X may be any of the amino acids described in the section “Amino acid X” herein below. Each β-strand peptide sequence comprises multiple amino acids Z and X. It is understood that the Zs within a β-strand sequence may be the same, partially the same or different amino acids. Similarly, the Xs within a β-strand sequence may be the same, partially the same or different amino acids.

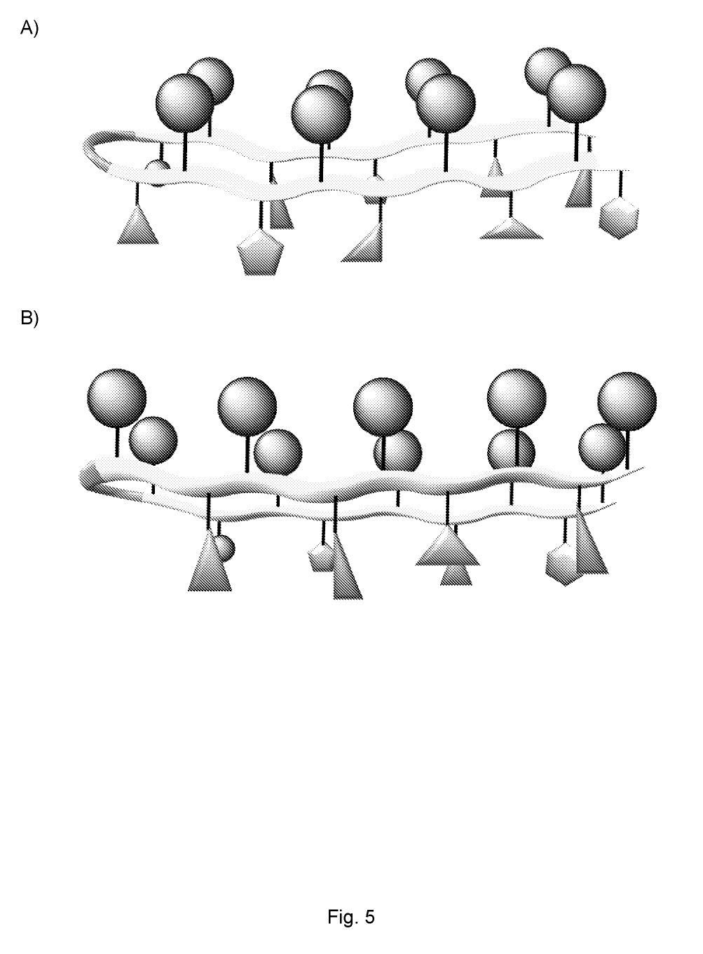

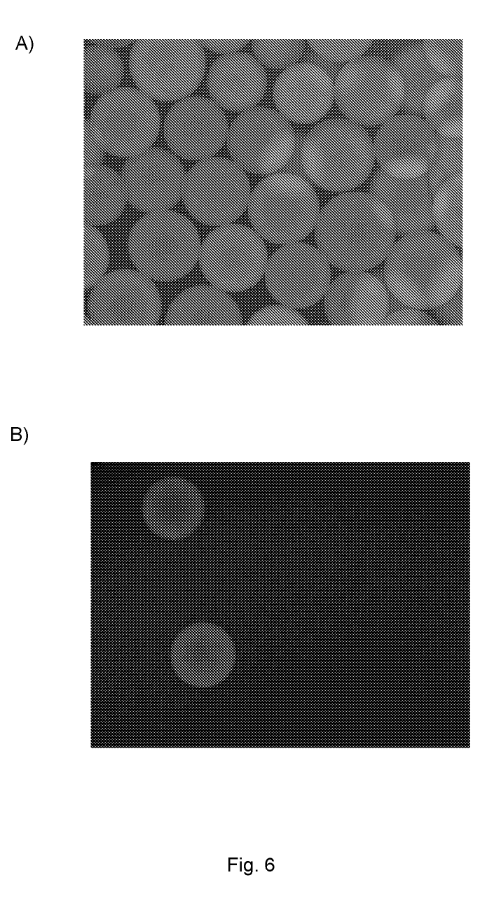

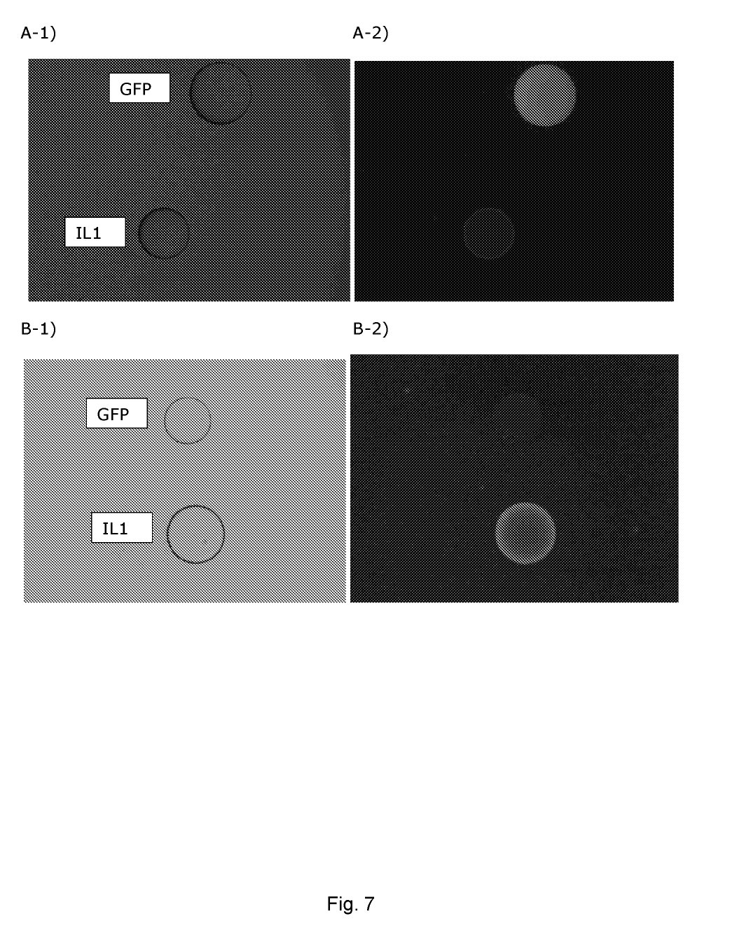

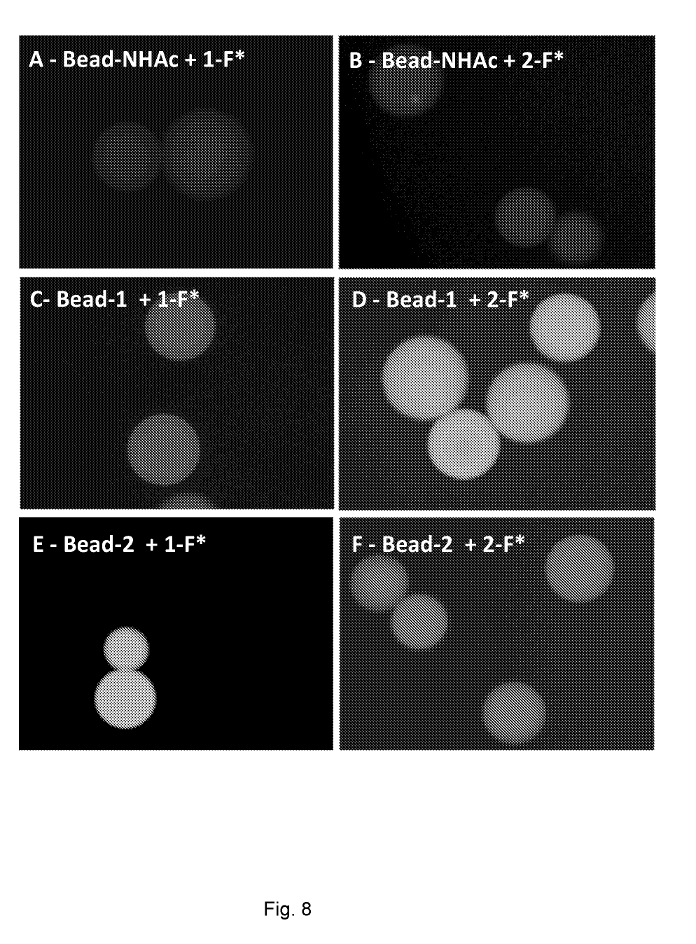

Thus the term (XZ)ₙ indicates a repetitive sequence of two types of amino acids, but not necessarily a repetitive sequence of the same two amino acids. In general the amino acids Z ensure the β-strand structure, and also provide solubility in water. Thus, typically, the β-body are water soluble, and thus useful as a diagnostic or therapeutic agent as described below. In general the side chains of all amino acids Z of a β-strand will be pointing in roughly the same direction, thereby taking part in a first surface of the β-body. Typically, all the side chains of all amino acids Z of a β-body will be pointing in roughly the same direction, thereby forming the first surface of the β-body. This may ensure stability of the 3-dimensional structure, e.g. the β-hairpin or the β-sheet of the β-body. A schematic example of a β-body is provided in FIG. 5, where the side chains of the amino acids Z are shown as balls.

Similarly, in general the side chains of all amino acids X of a β-strand will be pointing in roughly the same direction, thereby taking part in a second surface of the β-body. Preferably, the side chains of the amino acids Z will point in a different direction than the side chains of the amino acids X. Typically, all the side chains of all amino acids X of a β-body will be pointing in roughly the same direction, thereby forming a second surface of the β-body. The second surface of the β-body typically provides the binding specificity of the β-body. A schematic example of a β-body is provided in FIG. 5, where the side chains of the amino acids X are shown in various polygonic shapes.

In general all the side chains of all amino acids X of a β-body will be pointing in roughly the same direction, thereby forming an surface of the β-body. This may ensure stability of the 3-dimensional structure, e.g. the β-hairpin or the β-sheet of the β-body.

The β-strand peptide sequences are connected by β-turn sequences. Typically, the β-turn sequence introduces a bend in the peptide resulting in the two β-strands attached to a β-turn are positioned in proximity to each other in an anti-parallel arrangement. The β-turn may enable hydrogen bonding between the backbone amides in the two opposing strands.

Each β-turn sequence may individually have the following general sequence III:

X_{q}BUX_{q}

and may be any of the β-turn peptide sequences described herein below in more detail in the section “β-turn peptide sequences”. In embodiments of the invention wherein the β-body comprise more than one β-turn sequence, it is understood that even though the β-turn peptide sequences have the same general sequence, then each β-turn within a β-body may have different specific sequences.

The peptide sequences of the β-bodies of the invention are typically organised as alternating forward β-strand peptide sequences and reverse β-strand peptide sequences, wherein any two β-strand peptide sequences are separated by a β-turn peptide sequence.

Each β-body may comprise multiple β-strand peptide sequences. Whereas the β-body must comprise at least two β-strand sequences (typically a forward β-strand peptide sequence and a reverse β-strand peptide sequence), then there is in principle no upper limit for the number of β-strand peptide sequences, wherein the β-strand peptide sequences may be connected to each other via β-turns. It is however preferred that the β-strands peptide sequences of s β-body can form either a β-hairpin or a β-sheet. When the β-body comprise only two β-strand peptide sequences, then they typically form a β-hairpin, whereas in β-bodies comprising more than two, the β-strand peptide sequences preferably form a β-sheet.

In one embodiment the β-body may comprise in the range of 2 to 10 β-strand peptide sequences connected by β-turn peptide sequences. Said β-strand sequences are preferably alternating forward and reverse β-strand peptide sequences connected by β-turn peptide sequences.

In one embodiment the β-body may comprise in the range of 2 to 8, such as in the range of 2 to 6 β-strand peptide sequences connected by β-turn peptide sequences. Said β-strand sequences are preferably alternating forward and reverse β-strand peptide sequences connected by β-turn peptide sequences.

In one embodiment the β-body may comprise in the range of 2 to 4 β-strand peptide sequences connected by β-turn peptide sequences. Said β-strand sequences are preferably alternating forward and reverse β-strand peptide sequences connected by β-turn peptide sequences.

In one embodiment the 8-body may comprise or consist of the following structure:
forward β-strand sequence
β-turn peptide sequence
reverse β-strand sequence,

wherein the forward β-strand sequence and the reverse β-strand sequence are arranged as antiparallel β-strands.

In one embodiment, the β-body may comprise or consist of the following structure:
forward β-strand sequence
β-turn peptide sequence
reverse β-strand sequence
β-turn peptide sequence
forward β-strand sequence,

wherein the forward β-strand sequences and the reverse β-strand sequence are arranged as antiparallel β-strands.

In one embodiment, the β-body may comprise or consist of the following structure:
forward β-strand sequence
βturn peptide sequence
reverse β-strand sequence
β-turn peptide sequence
forward β-strand sequence
β-turn peptide sequence
reverse β-strand sequence.

wherein the forward β-strand sequences and the reverse β-strand sequences are arranged as antiparallel β-strands.

In one embodiment the β-body may be a compound comprising or consisting of a polypeptide having the general sequence XIX:

Xᵣ(ZX)ₘX_{q}PGX_{q}(XZ)ₙXᵣ,

wherein

each X individually may be any amino acid, e.g. any of the amino acids described herein below in the section “Amino acid X”, and each r individually is an integer in the range of 0 to 5, preferably in the range of 0 to 3, for example each r is 0 and wherein Z is as described herein below in the section “Amino acid Z”, preferably all Z except at the most 2, preferably at the most 1 are Thr, and wherein each q is an integer in the range of 0 to 3 as described herein below in more detail in the section “β-turn peptide sequences”.

In one embodiment the β-body may be a compound comprising or consisting of a polypeptide having the general sequence XVI:

Xᵣ(ZX)ₘPG(XZ)ₙXᵣ,

wherein

each X individually may be any amino acid, e.g. any of the amino acids described herein below in the section “Amino acid X”, and each r individually is an integer in the range of 0 to 5, preferably in the range of 0 to 3, for example each r is 0 and wherein Z is as described herein below in the section “Amino acid Z”, preferably all Z except at the most 2, preferably at the most 1 are Thr.

In one embodiment the β-body may be a compound comprising or consisting of a polypeptide having the general sequence XX:

Xᵣ(TX)ₘX_{q}PGX_{q}(XT)ₙXᵣ,

wherein

each X individually may be any amino acid, e.g. any of the amino acids described herein below in the section “Amino acid X”, and each r individually is an integer in the range of 0 to 5, preferably in the range of 0 to 3, for example each r is 0, and wherein each q is an integer in the range of 0 to 3 as described herein below in more detail in the section “β-turn peptide sequences”.

In one embodiment the β-body may be a compound comprising or consisting of a polypeptide having the general sequence IV:

Xᵣ(TX)ₘPG(XT)ₙXᵣ,

wherein

each X individually may be any amino acid, e.g. any of the amino acids described herein below in the section “Amino acid X”, and each r individually is an integer in the range of 0 to 5, preferably in the range of 0 to 3, for example each r is 0.

In one embodiment the β-body may be a compound comprising or consisting of a polypeptide having the general sequence XXI:

Xᵣ(ZX)ₘX_{q}PGX_{q}(XZ)ₙXX_{q}PGX_{q}X(ZX)ₘXᵣ

wherein

each X individually may be any amino acid, e.g. any of the amino acids described herein below in the section “Amino acid X”, and each r individually is an integer in the range of 0 to 5, preferably in the range of 0 to 3, for example each r is 0 and wherein Z is as described herein below in the section “Amino acid Z”, preferably all Z except at the most 3, preferably at the most 2, preferably at the most 1 are Thr, and wherein each q is an integer in the range of 0 to 3 as described herein below in more detail in the section “β-turn peptide sequences”.

In one embodiment the β-body may be a compound comprising or consisting of a polypeptide having the general sequence XVII:

Xᵣ(ZX)ₘPG(XZ)ₙXPGX(ZX)ₘXᵣ

wherein

each X individually may be any amino acid, e.g. any of the amino acids described herein below in the section “Amino acid X, and each r individually is an integer in the range of 0 to 5, preferably in the range of 0 to 3, for example each r is 0 and wherein Z is as described herein below in the section “Amino acid Z”, preferably all Z except at the most 3, preferably at the most 2, preferably at the most 1 are Thr.

In one embodiment the β-body may be a compound comprising or consisting of a polypeptide having the general sequence XXII:

Xᵣ(TX)ₘX_{q}PGX_{q}(XT)ₙXX_{q}PGX_{q}X(TX)ₘXᵣ

wherein

each X individually may be any amino acid, e.g. any of the amino acids described herein below in the section “Amino acid X, and each r individually is an integer in the range of 0 to 5, preferably in the range of 0 to 3, for example each r is 0, and wherein each q is an integer in the range of 0 to 3 as described herein below in more detail in the section “β-turn peptide sequences”.

In one embodiment the β-body may be a compound comprising or consisting of a polypeptide having the general sequence V:

Xᵣ(TX)ₘPG(XT)ₙXPGX(TX)ₘXᵣ

wherein

each X individually may be any amino acid, e.g. any of the amino acids described herein below in the section “Amino acid X, and each r individually is an integer in the range of 0 to 5, preferably in the range of 0 to 3, for example each r is 0.

In one embodiment the β-body may be a compound comprising or consisting of a polypeptide having the general sequence XXIII:

Xᵣ(ZX)ₘX_{q}PGX_{q}(XZ)ₙXX_{q}PGX_{q}X(ZX)ₘX_{q}PGX_{q}(XZ)ₙXᵣ

wherein

each X individually may be any amino acid, e.g. any of the amino acids described herein below in the section “Amino acid X, and each r individually is an integer in the range of 0 to 5, preferably in the range of 0 to 3, for example each r is 0 and wherein Z is as described herein below in the section “Amino acid Z”, preferably all Z except at the most 4, for example at the most 3, preferably at the most 2, preferably at the most 1 are Thr, and wherein each q s an integer in the range of 0 to 3 as described herein below in more detail in the section “β-turn peptide sequences”.

In one embodiment the β-body may be a compound comprising or consisting of a polypeptide having the general sequence XVIII:

Xᵣ(ZX)ₘPG(XZ)ₙXPGX(ZX)ₘPG(XZ)ₙXᵣ

wherein

each X individually may be any amino acid, e.g. any of the amino acids described herein below in the section “Amino acid X, and each r individually is an integer in the range of 0 to 5, preferably in the range of 0 to 3, for example each r is 0 and wherein Z is as described herein below in the section “Amino acid Z”, preferably all Z except at the most 4, for example at the most 3, preferably at the most 2, preferably at the most 1 are Thr.

In one embodiment the β-body may be a compound comprising or consisting of a polypeptide having the general sequence XXIV:

Xᵣ(TX)ₘX_{q}PGX_{q}(XT)ₙXX_{q}PGX_{q}X(TX)ₘX_{q}PGX_{q}(XT)ₙXᵣ

wherein

each X individually may be any amino acid, e.g. any of the amino acids described herein below in the section “Amino acid X, and each r individually is an integer in the range of 0 to 5, preferably in the range of 0 to 3, for example each r is 0, and wherein each q s an integer in the range of 0 to 3 as described herein below in more detail in the section “β-turn peptide sequences”.

In one embodiment the β-body may be a compound comprising or consisting of a polypeptide having the general sequence VI:

Xᵣ(TX)ₘPG(XT)ₙXPGX(TX)ₘPG(XT)ₙXᵣ

wherein

each X individually may be any amino acid, e.g. any of the amino acids described herein below in the section “Amino acid X, and each r individually is an integer in the range of 0 to 5, preferably in the range of 0 to 3, for example each r is 0.

As described herein elsewhere amino acids may be named using the IUPAC one-letter code or 3-letter code. Thus, T and P in respect of the general sequences IV, V and VI are threonine and proline, respectively. Said threonine and proline may be either in D or L configuration. As described herein elsewhere it is preferred that all amino acids within a single β-strand are of the same either D or L configuration.

In respect of general sequences I, IV, V, VI, XVI, XVII, XVIII, XIX, XX, XXI, XXII, XXIII or XXIV m may individually be any integer, typically an integer of at least 2, preferably an integer of at least 3, for example an integer in the range of 3 to 12, such as an integer in the range of 3 to 7, for example an integer in the range of 3 to 5. It is understood that in embodiments of the invention relating to β-bodies comprising several forward β-strand peptide sequences or several (TX)ₘ sequences, then m may be the same or different integers in relation to each forward β-strand peptide sequence and each (TX)ₘ sequence. In one embodiment it may be preferred that all m within one β-body are in a range of +/−2 of each other. For example all m within one β-body may be in a range of +/−1 of each other or they may be identical.

In respect of general sequences II, IV, V, VI, XVI, XVII, XVIII, XIX, XX, XXI, XXII, XXIII or XXIV n may individually be any integer, typically an integer of at least 2, preferably an integer of at least 3, for example an integer in the range of 3 to 12, such as an integer in the range of 3 to 7, for example an integer in the range of 3 to 5. It is understood that in embodiments of the invention relating to β-bodies comprising several reverse β-strand peptide sequences or several (XT)ₙ sequences, then n may be the same or different integers in relation to each reverse β-strand peptide sequence and each (XT)ₙ sequence. In one embodiment it may be preferred that all n within one β-body are in a range of +/−2 of each other. For example all n within one β-body may be in a range of +/−1 of each other or they may be identical.

In one embodiment all m and n within one β-body may be in a range of +/−2 of each other. For example all m and n within one β-body may be in a range of +/−1 of each other or they may be identical.

Compared to conventional antibodies, the β-bodies of the invention are typically small molecules. Typically they consist of in the range of 10 to 100 amino acids, such as in the range of 15 to 50 amino acids, for example in the range of 15 to 25 amino acids. In one embodiment, the the β-bodies of the disclosure comprise or consist of a sequence selected from the group consisting of SEQ ID NO: 1 to SEQ ID NO: 61.

It is understood that the β-bodies according to the invention also may be cyclic. Thus, the most N-terminal amino acid of the β-body may be covalently linked to the most C-terminal amino acid of the β-body. In other words, the β-body may be a cyclic peptide. Whereas the sequences provided herein are provided in a linear format, it is understood that the peptides having these sequences may also be cyclic. Thus, for example the β-body may be a cyclic peptide comprising or consisting of any of the general sequences I, IV, V, VI, XVI, XVII or XVIII.

In a preferred embodiment the β-body is however linear, for example a linear peptide comprising or consisting of any of the general sequences I, IV, V, VI, XVI, XVII or XVIII.

In one embodiment the β-body is an inward β-body, wherein the Z amino acid residue as defined in the section below “Amino acid Z”, for example a threonine, immediately precedes and follows a β-type2-turn.

In one embodiment the β-body is an outward β-body, wherein the recognition residues, such as the X amino acid residue as defined in the section below “Amino acid X”, immediately precede and follows a β-type2-turn.

Forward β-Strand Peptide Sequences

The present invention relates to compounds comprising forward β-strand peptide sequences. The forward β-strand peptide sequence typically has the following general sequence I:

(ZX)ₘ

In respect of forward β-strand peptide sequences of general sequence I, then each Z individually may be any of the amino acids described herein below in the section “Amino acid Z”. Thus, typically each Z may individually be selected from the group consisting of polar β-branched amino acids and strand bridging amino acid. Whereas amino acid Z in general may contribute to the 3-dimensional structure of the β-body it is acceptable that at the most one Z within each forward β-strand peptide sequence may be any amino acid. Thus, at the most one Z within each forward β-strand sequence may be an amino acid, which is not a β-branched or strand bridging amino acid. It is understood that the Zs within a forward β-strand peptide sequence may be the same, partially the same or different amino acids.

In one preferred embodiment of the invention at least one, for example all forward β strand sequences within a β-body may have the following general sequence VII:

(TX)ₘ

wherein T is threonine.

In respect of forward β-strand peptide sequences of general sequences I and VII, then each X individually may be any amino acid, e.g. any of the amino acids described herein below in the section “Amino acid X. It is understood that each X within a forward β-strand peptide sequence may all be the same, partially the same or different amino acids.

In respect of forward β-strand peptide sequences of general sequences I and VII, then m may be any integer, typically an integer of at least 2, preferably an integer of at least 3, for example an integer in the range of 3 to 12, such as an integer in the range of 3 to 7, for example an integer in the range of 3 to 5.

The amino acids Z, X and T of the general sequences I and VII may be of either D or L configuration. It is preferred that all amino acids within one β-strand peptide sequence all have the same either D or L configuration. Thus, in some embodiments of the invention all amino acids Z and X of the general sequence I are of D-configuration. In some embodiments of the invention all amino acids Z and X of the general sequence I are of L-configuration. Thus, in some embodiments of the invention all amino acids T and X of the general sequence VII are of D-configuration. In some embodiments of the invention all amino acids T and X of the general sequence VII are of L-configuration.

In some embodiments, the forward β-strand peptide sequence of the β-body may be covalently linked to the reverse β-strand peptide sequence of the same β-body and form a cyclic β-body.

In some embodiments, the forward β-strand peptide sequence and the reverse β-strand peptide sequence of the β-body may both comprise a non-proteogenic amino acid residue, and the β-body is in a cyclic form.

Reverse β-Strand Peptide Sequences

The present invention relates to compounds comprising reverse β-strand peptide sequences. The reverse β-strand peptide sequence typically has the following general sequence II:

(XZ)ₙXᵣ

In respect of reverse β-strand peptide sequences of general sequence II, then each Z individually may be any of the amino acids described herein below in the section “Amino acid Z”. Thus, typically each Z may individually be selected from the group consisting of polar β-branched amino acids and strand bridging amino acid. Whereas amino acid Z in general may contribute to the 3-dimensional structure of the β-body it is acceptable that at the most one Z within each reverse β-strand peptide sequence may be any amino acid. Thus, at the most one Z within each reverse β-strand sequence may be an amino acid, which is not a β-branched or strand bridging amino acid. It is understood that the Zs within a reverse β-strand peptide sequence may be the same, partially the same or different amino acids.

In one preferred embodiment of the invention at least one, for example all reverse β strand sequences within a β-body may have the following general sequence VIII:

(XT)ₙ

wherein T is threonine.

In another preferred embodiment of the invention at least one, for example all reverse β strand sequences within a β-body may have the following general sequence IX:

(XT)ₙXᵣ

wherein T is threonine. In respect of reverse β-strand peptide sequences of general sequences II, VIII and IX, then each X individually may be any amino acid, e.g. any of the amino acids described herein below in the section “Amino acid X. It is understood that the Xs within a reverse β-strand peptide sequence may be the same, partially the same or different amino acids.

In respect of reverse β-strand peptide sequences of general sequences II, VIII and IX, then n may be any integer, typically an integer of at least 2, preferably an integer of at least 3, for example an integer in the range of 3 to 12, such as an integer in the range of 3 to 7, for example an integer in the range of 3 to 5.

In respect of reverse β-strand peptide sequences of general sequences II and IX, then r may be any integer, typically an integer of at the most 3, for example an integer in the range of 0 to 3. Thus, r may for example be 0 or 1.

The amino acids Z, X and T of the general sequences II, VIII and IX may be of either D or L configuration. It is preferred that all amino acids within one reverse β-strand peptide sequence all have the same either D or L configuration. Thus, in some embodiments of the invention all amino acids Z and X of the general sequence II are of D-configuration. In some embodiments of the invention all amino acids Z and X of the general sequence II are of L-configuration. Thus, in some embodiments of the invention all amino acids T and X of the general sequence VIII or IX are of D-configuration. In some embodiments of the invention all amino acids T and X of the general sequence VIII or IX are of L-configuration.

β-Turn Peptide Sequences

The present invention relates to compounds comprising a β-turn peptide sequence. The β-turn peptide sequence typically has the following general sequence III:

X_{q1}BUX_{q2}

In some embodiments the invention B is proline. Thus, at least one, for example all β-turn peptide sequences within a β-body may have the following general sequence X:

X_{q1}PUX_{q2}.

In some embodiments the invention U is glycine. Thus, at least one, for example all β-turn peptide sequences within a β-body may have the following general sequence XI:

X_{q1}BGX_{q2}.

In some embodiments the invention at least one, for example all β-turn peptide sequences within a β-body may have the following general sequence XII:

X_{q1}PGX_{q2}.

In respect of β-turn peptide sequences of general sequences III and X, then U may be any of the amino acids U described herein below in the section “Amino acid U”.

In respect of β-turn peptide sequences of general sequences III and XI, then B for example be selected from the group consisting of proline, substituted proline and pipecolic acid. Substituted proline may for example be proline substituted with a substituent selected from the group consisting of —OH, —NH₂, —O—R, —NH—R, —NR₂, halogen and C₁₋₃-alkyl, where R is alkyl, acyl or a peptide. In one embodiment B may be selected from the group consisting of Pro, hydroxyproline (Hyp), 4-amino-Pro and pipecolic acid.

It is preferred that q1 and q2 are selected to ensure that the amino acid Zs of the forward β-strand are positioned opposite the Zs of the reverse β-strand, so that the side chains of the amino acids Z are located on opposing positions on the same surface of the β-body. This may be obtained by ensuring that the relation between q1 and q2 is such that q1−q2=−4, −2, 0, 2 or 4. The term “q1−q2” as used herein refers to “q1 minus q2”.

In respect of β-turn peptide sequences of general sequences III, X, XI and XII, then each q1 and q2 may individually be integers, preferably an integer of at the most 5, such as an integer in the range of 0 to 5, such as an integer in the range of 0 to 3. The relation between q1 and q2 is preferably such that q1−q2=−4, −2, 0, 2 or 4, and preferably q1−q2=0. Thus, q1 and q2 may for example both be 0 or both may be 1. It is understood that q1 and q2 within a β-body may be the same or different integers.

In one embodiment at least one, for example all β-turn peptide sequences within a β-body may have the following general sequence XIII:

XPGX.

In one embodiment at least one, for example all β-turn peptide sequences within a β-body may have the following general sequence XIV:

PGX.

In one embodiment at least one, for example all β-turn peptide sequences within a β-body may have the following general sequence XV:

XPG.

In respect of β-turn peptide sequences of the general sequences III, X, XI, XII, XIII, XIV and XV then each X individually may be any amino acid, for example any of the amino acids described herein below in the section “Amino acid X”.

In one embodiment at least one, for example all β-turn peptide sequences within a β-body may have the following sequence:

PG

As described herein elsewhere amino acids may be named using the IUPAC one-letter code. Thus, P and G are proline and glycine, respectively.

The amino acids X, B, U, P and G of the general sequences III, X, XI, XII, XIII, XIV and XV may be of either D or L configuration.

Amino Acid X

The present invention relates to compounds comprising peptide sequences comprising one or more amino acids X.

The amino acid X may be any amino acid, β-amino acid or γ-amino acid, such as any compound of the following general structure I:

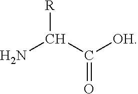

When the amino acid X is part of a peptide sequence, the amino acid is linked to neighbouring amino acids via peptide bonds.

In one embodiment the amino acid X may be a proteinogenic amino acid, i.e. any amino acid which is incorporated into proteins.

In one embodiment the amino acids X may be the enantiomeric D-form of a proteinogenic amino acid, i.e. any D-amino acid corresponding to L-amino acids, which are incorporated into proteins. Preferably, a given β-body only comprises amino acids of either the D-form or of the L-form. However, a β-body may comprise amino acids which are all L-form or all D-form, wherein 1 to 3 amino acids may have the opposite configuration, i. e. an L-β-body can contain 1 to 3 D-amino acids and vice-versa.

In one embodiment, one or more of the amino acids X, e.g. all amino acid X may be selected from the group consisting of Glycine, Alanine, α-Amino-n-butyric acid, Norvaline, Valine, Norleucine, Leucine, Isoleucine, Alloisoleucine, t-leucine, α-Amino-n-heptanoic acid, Proline, Pipecolic acid, α,β-diaminopropionic acid, α,γ-diaminobutyric acid, Ornithine, lysine, Aspartic acid, Glutamic acid, Serine, Threonine, Allothreonine, Methionine, Homocysteine, Homoserine, β-Alanine, β-Amino-n-butyric acid, β-Aminoisobutyric acid, γ-Aminobutyric acid, α-Aminoisobutyric acid, isovaline, Sarcosine, N-ethyl glycine, N-propyl glycine, N-isopropyl glycine, N-methyl alanine, N-ethyl alanine, N-methyl β-alanine, N-ethyl β-alanine, isoserine, α-hydroxy-γ-aminobutyric acid, propargylglycin and 4-azido-2-aminobutanoic acid.

In order to improve β-sheet integrity, it may be preferred that each β-body comprise at the most 2, e.g. at the most one 13- or γ-amino acids. Preferably, said β- or γ-amino acid is positioned in the β-turn or at the ends of the β-body.

Preferably, amino acid X is not N-alkylated. Frequently, the nitrogen atoms of the amino acids X positioned in the β-strands may be involved in interstrand hydrogen bonds

In one embodiment one or more amino acid X may be selected from the group of substituted glycines. Substituted glycine residues preferably have the general formula —NH—CHR—CO—, where R may be selected from the group consisting of linear C₁-C₂₀-alkyl, branched C₁-C₂₀-alkyl groups., aryl, and substituted alkyl. Said branched C₁-C₂₀-alkyl group may preferably be selected from the group consisting of iPr, iBu, tBu, sBu, pent-2-yl, pent-3-yl and 2,2-dimethylpropyl. Said substituted alkyl may preferably be C₁₋₂₀-alkyl substituted with one or more substituents. For example substituted alkyl may be selected from the group consisting of benzyl, allyl, propargyl, aryl-alkyl, hydroxyalkyl, aminoalkyl, sulfhydrylalkyl alkylaminoalkyl, dialkylaminoalkyl, alkoxyalkyl, alkylthioalkyl, sulfonylalkyl. Substituted alkyl may also be selected from the group consisting of benzyl, C₁-C₂₀-allyl, propargyl, aryl- C₁-C₂₀-alkyl, C₁-C₂₀-hydroxyalkyl, C₁-C₂₀-aminoalkyl, C₁-C₂₀-sulfhydryl-alkyl, C₁-C₂₀-alkylaminoalkyl, C₁-C₂₀-dialkylaminoalkyl, C₁-C₂₀-alkoxyalkyl, C₁-C₂₀-alkylthioalkyl, sulfonyl-C₁-C₂₀-alkyl. The substituted alkyl groups may also be substituted with charged groups, such as phosphates sulfonates, sulphates, carboxylates, ammonium and guanidyl groups.

In one embodiment one or more amino acid X may be selected from the group of disubstituted glycines. Disubstituted glycine residues preferably have the general formula —NH—CR₁R₂—CO—, where R₁ and R₂ individually are selected from the group consisting of linear C₁-C₂₀-alkyl, branched C₁-C₂₀-alkyl and aryl. Said branched alkyl may in particular be selected from the group consisting of iPr, iBu and tBu.

In one embodiment, in the range of 1 to 4 of the amino acids X within a given β-body may be selected from the group of β- and γ-amino acids, e.g. β- and γ-amino acids analogous to the aforementioned amino acids.

In one embodiment, one or more of the amino acid X may be selected from the group consisting of proteinogenic amino acids and non-proteinogenic amino acids, wherein the non-proteinogenic amino acids are selected from the group of amino acids consisting of a-amino-n-butyric acid, norvaline, norleucine, alloisoleucine, t-leucine, α-amino-n-heptanoic acid, α,β-diaminopropionic acid, α,γ-diaminobutyric acid, ornithine, allothreonine, homocysteine, homoserine, α-aminoisobutyric acid, isovaline, sarcosine, homophenylalanine, propargylglycin, 4-azido-2-aminobutanoic acid and the D-form of any of the proteinogenic amino acids. Aforementioned non-proteinogenic amino acids may be either in the D-form or the L-form.

Proteinogenic amino acids may in particular be amino acids selected from the group consisting of Alanine, Cysteine, Aspartic acid, Glutamic acid, Phenylalanine, Glycine, Histidine, Isoleucine, Lysine, Leucine, Methionine, Asparagine, Pyrrolysine, Proline, Glutamine, Arginine, Serine, Threonine, Selenocysteine, Valine, Tryptophan and Tyrosine.

In one embodiment one or more of the amino acid X, e.g. all of the amino acids X may be selected from the group of standard amino acids. Thus, in one embodiment, at least 70%, such as at least 80%, for example at least 90%, such as all X of a β-body are standard amino acids.

The amino acid X may generally be of L or of D-configuration. In one embodiment all amino acids X within one β-strand peptide sequence are of the D-configuration. In one embodiment all amino acids X within one β-strand sequence are in the L-configuration. Thus, in one embodiment the amino acid X may be an amino acid corresponding to any of the standard amino acids, but in D configuration. Thus, in one embodiment, at least 70%, such as at least 80%, for example at least 90%, such as all X of a β-body are corresponding to standard amino acids, but are in the D-configuration.

In one embodiment it may be preferred that all amino acids within an β-body is either in the D-configuration or the L-configuration. Thus, in one embodiment all amino acids X and all amino acids Z of all β-strand sequences within a β-body is in the D-configuration. In another embodiment all amino acids X and Z within all β-strand peptide sequences of a β-body are of the L-configuration.

Amino Acid Z

The present invention relates to compounds comprising peptide sequences comprising a plurality of amino acid Z.

Amino acid Z is preferably an amino acid, which can contribute the 3-dimensional structure of the β-bodies. In particular, each amino acid Z may individually be selected from the group consisting of Thr, polar β-branched amino acids, non-proteninogenic α-branched amino acids that promote β-strand structure and strand bridging amino acids.

Furthermore, within each β-strand at the most two amino acids Z, for example at the most one amino acid Z may be any amino acid. Accordingly up to two amino acids Z, e.g. up to one amino acid Z within each β-strand may be an amino acid, which is not Thr, polar β-branched amino acids and strand bridging amino acids.

Thus, in one embodiment one or more amino acids Z are β-branched amino acids. Thus, one or more amino acids Z may be selected from the group consisting of isoleucine, threonine, allothreonine, alloisoleucine valine, 2-aminoisobutyric acid, 2-amino-3,3-dimethylbutanoic acid, propargylglycine and 4-azido-2-aminobutanoic acid.

In one embodiment one or more amino acids Z are non-proteinogenic α-branched amino acids that promote β-strand structure, such as an amino acid selected from the group consisting of α-aminoisobutyric acid, diethylglycine, dipropylglycine, diphenylglycine, 1-aminocyclobutane-1-carboxylic acid, 1-aminocyclopentane-1-carboxylic acid, 1-aminocyclohexane-1-carboxylic acid, 1-aminocycloheptane-1-carboxylic acid, propargylglycine and 4-azido-2-aminobutanoic acid.

In one embodiment one or more amino acids Z are strand bridging amino acids. Thus, one or more amino acids Z may be selected from the group consisting of cysteine, asparagine, threonine, aspartic acid, glutamic acid, β-amino alanine, γ-amino-α-aminobutyric acid, ornitine, lysine, amino acids substituted with alkyne, amino acids substituted with azide and amino acids suitable for bridging by reductive amination. Amino acids substituted with either alkyne or azide are preferably such amino acids, which are useful for click chemistry e. g. propargyl glycine, β-azidoalanine, γ-azido-α-aminobutyric acid or 4-azido-2-aminobutanoic acid. Amino acids suitable for bridging by reductive amination include amino acid aldehydes, for example aldehydes generated from e. g. 2-allyl-glycine or 2-homoallyl-glycine through dihydroxylation/oxidation.

In one embodiment one or more of the amino acids Z may be N-alkylated with any linear C₁-C₂₀-alkyl , branched C₁-C₂₀-alkyl, or with substituted alkyl groups. Said substituted alkyl may for example be substituted C₁-C₂₀-alkyl, such as benzyl, allyl, propargyl, azidoalkyl, aminoalkyl, sulfhydrylalkyl or a haloalkyl, wherein any of the aforementioned preferably is substituted C₁₋₂₀-alkyl. Said branched C₁-C₂₀-alkyl, may for example be iPr, iBu, or tBu.

In a preferred embodiment at least some of the amino acids Z are threonine. Accordingly, at least 70%, such as at least 80%, preferably at least 90%, such as at least 95% of the amino acids Z within each β-strand peptide sequences may be threonine. It is also preferred that at least 70%, such as at least 80%, preferably at least 90%, such as at least 95% of the amino acids Z within a β-body may be threonine.

In one embodiment all amino acids Z within a β-body are threonine.

The amino acid Z may be either in the L or the D-configuration. Thus, in one embodiment all amino acids Z within one β-strand peptide sequence are of the D-configuration. In another embodiment all amino acids Z within one β-strand sequence are in the L-configuration.

Thus, in one embodiment at least some, for example all amino acids Z within a β-body are L-threonine. In another embodiment at least some, for example all amino acids Z within a β-body are D-threonine.

Amino Acid U

The present invention relates to compounds comprising peptide sequences comprising an amino acid U.

The amino acid U is typically a relatively small amino acid, which when positioned next to proline may aid in the formation of a β-turn. In particular, each amino acid U may individually be an amino acid of the general structure VI:

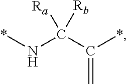

wherein Rₐ and R_{b} individually are selected from the group consisting of —H and C₁₋₆-alkyl, wherein Rₐ and R_{b} may be linked to form a cyclic structure. If Rₐ is different from R_{b} the amino acid U may be of either S or L configuration.

In one embodiment the amino acid U is glycine. The amino acid U may be of the S or R configuration. The amino acid U may for example be D-glycine or L-glycine.

Method of Preparing β-Bodies

The β-bodies according to the present invention comprise or consist of a plurality of linked peptide sequences. Accordingly, the β-body comprises or even consists of a polypeptide.

Accordingly, the β-bodies can be prepared by standard methods for producing polypeptides.

In one embodiment, the β-bodies of the invention are prepared by standard chemical peptide synthesis, for example by Solid-phase peptide synthesis (SPPS).

Typically such methods involve use of a solid support attached to a linker on which peptide chains can be built. During synthesis the β-body will remain covalently attached to the solid support, and may then optionally be cleaved from the solid support once synthesis is complete. Thus, the linker may be a cleavable linker.

The SPPS usually comprise several cycles of reacting the free N-terminal amine of the peptide associated with the solid support, with an N-protected amino acid. The cycles are ordered so that the sequence of amino acids can be controlled. SPPS usually proceeds in a C-terminal to N-terminal fashion. Accordingly, the method may comprise the steps of:
i. providing a solid support attached to a linker comprising free amino group
ii. providing the first amino acid of the β-body polypeptide sequence in the form of an N-protected amino acid,
iii. reacting said free amino group with said amino acid thereby forming a peptide bond
iv. deprotecting the amino group the linked amino acid thereby preparing a free amino group
v. washing away free reactants
vi. providing the next amino acid of the β-body polypeptide sequence in the form of an N-protected amino acid
vii. reacting the free amino group with said amino acid thereby forming a peptide bond
viii. Repeating steps iv. to vii until the entire polypeptide has been produced
ix. Optionally cleaving the linker.

The N-protected amino acids may for example be protected by Fmoc or Boc. The SPPS may be performed either manually or with the aid of automated synthesizers.

The solid support may be any useful solid support, for example be selected the solid support may be selected from the group consisting of polystyrene resin, polyamide resin, PEG hybrid polystyrene resin and PEG based resin. In particular, the solid support may be in the form of resin beads, e.g. PEGA beads. These beads could be encoded with micro-particles and may for example be any of the resin beads described in Meldal and Christensen, 2010.

A cyclization through click formation of a triazole or a disulfide bond may be desired to increase β-structure stability and selectivity. For example, two opposing threonine residues may be replaced with either a L-propargylglycin (Pra) and L-4-azido-2-aminobutanoic acid (Abu(N₃)) residue for copper(I)-catalysed azide alkyne cycloaddition (CuACC) reaction or with two cysteine residues for disulphide formation. Two threonine residues close to the open end of the β-body may beneficial be chosen to form a cyclic structure. Several threonine pairs may be tested to identify the pairs that pose the least influence on the fitted β-body structure. Alternatively, the cyclization can be implemented already at the stage of the degenerate β-body described above. The possibility of adding an elongation probe to the β-body may also be evaluated. It may be beneficial to add an elongation probe to link the β-body to an additional moiety as described in the section “Conjugated moiety”, as well as a probe, a peptide, or a solid support. For example, elongating the β-body with residues such as glycine, alanine and/or serine may be beneficial to the functionality of the β-body. Elongating the β-body with residues such as arginine and/or lysine may be beneficial to improve the solubility of the β-body.

In embodiments of the invention, where the β-body is linked to a conjugated moiety the β-body may be synthesized as described above and equipped with an extra amino acid building block containing a suitable click partner and upon purification it may be a moiety linked to attached to the appropriate partner for the click reaction. Said moiety may be an (encoded) biocompatible resin or another surface. The click partner may for example be tetrazine, aldehyde, aminoxy-group, azide or alkyne.

Alternatively, the β-bodies may be prepared using recombinant methods involving a nucleic acid encoding the polypeptide of the β-bodies. Such methods are also well known in the art and may involve the following steps:
Providing a host cell comprising a heterologous nucleic acid encoding the polypeptide of the β-body
Incubating said host cell under condition allowing growth of the host cell
Optionally purifying the β-body from the host cell

The β-bodies may also be prepared in vitro, for example by a method involving the steps of
Providing a nucleic acid encoding the polypeptide of the β-body
Providing reagents capable of transcription and translation of said nucleic acid
Incubating said nucleic acid with said reagents.

Method of Identifying β-Bodies

As described herein the invention relates to β-bodies, which are capable of specifically binding a target compound. Whereas the amino acids Z, B and U of the β-bodies enable a stable 3-dimensional structure of the β-body, e.g. in the form of a β-hairpin or β-sheet, then the amino acids X may be any amino acids and determine the specificity of the ⊐-body. Thus, the amino acids X are chosen to enable specific binding between the β-body and a target compound. The target compound may be any compound, for example another β-body, a peptide, an oligosaccharide or a protein.

There are several methods of identifying a β-body binding a target compound of interest. In embodiments of the invention, wherein the target compound is a protein, the target compound may be referred to as “protein of interest” or POI.

Method of Identifying β-Bodies

As described herein the invention relates to β-bodies, which are capable of specifically binding a target compound. Whereas the amino acids Z, B and U of the β-bodies enable a stable 3-dimensional structure of the β-body, e.g. in the form of a β-hairpin or β-sheet, then the amino acids X may be any amino acids and determine the specificity of the β-body. Thus, the amino acids X are chosen to enable specific binding between the β-body and a target compound. The target compound may be any compound, for example another β-body, a peptide, an oligosaccharide or a protein.

There are several methods of identifying a β-body binding a target compound of interest. In embodiments of the invention, wherein the target compound is a protein, the target compound may be referred to as “protein of interest” or POI.

If the 3-dimensional structure of the target compound is known, the methods may be computer based methods for identifying β-bodies structurally fitting a site on the target compound. The structure of the target compound may be determined e.g. by x-ray crystallography or NMR, or it may be publicly available e.g. in public databases such as PDB (http://www.rcsb.org/pdb/home/home.do) or PSILO (http://www.chemcomp.com/PSILO-Protein_Structure_Database_System.htm).

Thus, the method for identifying a β-body, wherein said β-body is capable of binding a target compound may comprise the steps of
a. Providing atom coordinates of a spatial structure representation of the target compound in a computer;
b. Providing said spatial structure with an electrostatic VDW surface representing the surface topology and charge distribution;
c. Generating spatial structure representations of a plurality of β-bodies according to the invention in the computer;
d. Selecting β-bodies fitting at least part of the spatial structure of the target compound in said computer;
e. Providing the spatial structure representations of said selected β-bodies with an electrostatic VDW surface representation;
f. Using molecular dynamics calculations to select the β-body with optimal complementarity of both surface topology and charge-charge interactions in said computer

thereby identifying a β-body capable of binding the target compound.

The method may be performed with the aid of any useful software, for example using Molecular Operating Environment (e.g MOE—ver.2015.10) from Chemical Computing Group. Another example of software useful in performing the method is Rosetta™. Typically step a. comprises loading information on the structure of the target compound to the computer, e.g. in the form of a PDB-file. Once a spatial structure representation is available it may be modified, e.g. by addition of hydrogen atoms and/or by investigation of the structure and correction of any missing parts e. g. by homology modelling or by restrained dynamics if possible. Preferably the modelling does not perturbate the sections correctly obtained from the structure.

Steps b. and c. of the method may be performed as below.

Once a spatial structure representation is available the model may be fixed in space and equipped with a molecular electrostatic surface in the computer.

Spatial structures of β-bodies may be prepared using a random library of β-bodies. A spatial structures of β-bodies may however also be prepared by preparing a spatial structure of a reference β-body. The reference β-body may be any β-body, e.g. any of the β-bodies described herein above in the section “β-body”. For example it may be a β-body according to any of the general sequences IV, V or VI described in that section. For the sake of simplicity all amino acids X of the reference β-body may be set to be the same amino acid, and preferably an amino acid lacking very distinct chemical features, e.g. Ala. Thus, the reference β-body may be a β-body of the general sequence (TX)ₘPG(XT)ₙ, in which X initially may be alanine, and wherein up to 30% of the threonines can be randomly replaced either with other β-branched or strand bridging amino acids.

The best fit between the reference β-body and the target compound is found. This may be done manually and/or by computer aided means, by moving and/or rotating the spatial structure representation of the reference β-body across the surface of the target compound to identify the sites for optimal interaction e.g. in terms of overall shape fitting and presence of grooves, pits and patches promising for interaction with amino acid side-chains.

To further improve affinity to the target compound, a degenerate inward β-body, wherein threonine immediately precedes and follow a β-type2-turn, or an outward β-body, wherein the recognition residues immediately precede and follow a β-type2-turn, wherein both may comprise or consist of D- and/or L-amino acids, may be selected and imported into MOE for a cleft (e.g. an active site) or surface recognition, respectively.

While maintaining the target compound in a fixed position, the β-body may beneficially be soaked in a drop of water (in silico), where it may be kept through the rest of the calculation process, and subject shortly (of 0.1 fs to 10 ns) to a temperature of 273 K to fit the overall structure to the shape of the surface.

The most promising positions of the reference β-body may be selected, e.g. the 1 to 10 most promising positions may be selected. Then best amino acid X are determined each amino acid X of the β-body in order to obtain the best fit for each side-chain into the selected binding side. The surface contact is optimized in terms of both topology and electrostatic potential. Thus, if all amino acids X of the reference β-body are alanine, then the best replacement for the alanine side-chain is determined. The residue replacement most likely to improve the contact is performed typically using a torsional angle of the α-β bond of 180° to either N or CO of the backbone. Only in rare cases or with β-branched residues is the option of 60°,−60° used. The replacement is followed by 50 ps MD-calculation to assess whether the affinity or fitting is improved.

During this process many rounds of fitting using molecular dynamics by annealing may be performed. Typically, in the range of 1 to 20 rounds, such as in the range of 1 to 10 rounds are performed to find the best fit. At each round the interaction may be evaluated and residues that may show good contact and match of electrostatic potential but which may prevent other residues from reaching the protein surface are identified. The fitting using molecular dynamics by annealing may be done at any useful temperature, frequently at a temperature in the range of 450-300 K, for in the range of 0.1 fs to 10 ns. The fitting may be done with 8-10 layers of added layers of water and may be performed to take into account the additive effects of amino acid side-chain orientation, H-bond network, hydrophobic interaction, charge-charge interaction.

Once a rough model of a useful β-body is designed, then residues of the target compound in direct contact with the β-body may be released from fixation. Thus, when the target compound is a protein the amino acids in the POI in direct contact with the β-body may be released from fixation, while the rest of the POI structure may remain fixed. Additional exchange of amino acids X may be performed to refine the interaction.

Once a useful β-body has been designed the interaction may be tested by molecular dynamics by annealing at a temperature in the range of 450 to 300 K for in the range of 1-2 ns. β-bodies showing stable interaction with the target compound under these conditions are selected.

The methods may also comprise a step of optimising a β-body. Optimisation may for example comprise scanning the β-body by replacement of each amino acid (e.g. each amino acid X) with another amino acid, e.g. Ala thereby obtaining a group of potentially optimised β-bodies. Optimisation may also comprise scanning the β-body by replacement of one amino acid, e.g. an amino acid X with several other amino acids, thereby obtained a group of potentially optimised β-bodies. The potentially optimised β-bodies may then be tested for improved properties, e.g. for having a lower annealing temperature. Such test may be performed using the computer models as described above or it may be tested in the laboratory.

In absence of a target crystal structure test β-bodies may be synthesized as a library or expressed in phage display libraries with variation of the recognition residues, followed by screening towards the target protein or other bio-surface. One-bead one-compound synthetic libraries may advantageously be used, for example those generated through split-mix approach with the structural turn residues and the threonine maintained. Phage display libraries may be panned against the target protein in the usual manner to generate high affinity ligands deciphered through DNA-sequencing.

Once a useful β-body has been designed it may be produced as described herein above in the section “Method of preparing β-bodies” and tested. Thus, the method for identification may further comprise the following steps
d. Providing a β-body of the spatial structure identified in step c.
e. Providing the target compound
f. Determining whether said β-body is capable of binding said target compound
g. Selecting β-bodies capable of binding said target compound.

The β-body may also be identified by selection from a library of putative β-bodies. Thus, the method for identifying a β-body capable of binding a target compound may comprise the steps of
Providing the target compound
Providing library comprising a plurality of test β-bodies, e.g. any of the β-bodies described in the section “β-body” herein above
Determining whether said test β-bodies are capable of binding said target compound
Selecting β-bodies capable of binding said target compound. thereby identifying a β-body capable of binding the target compound.

In order to facilitate handling of the library, the β-bodies may be immobilised on solid supports. Said solid supports may be any useful solid support including e.g. polystyrene resin, polyamide resin, PEG hybrid polystyrene resin or PEG based resin. In one embodiment, the test β-bodies are linked to solid supports in a manner so that each type of β-body is spatially separated from other types of β-bodies. For example the β-bodies may be immobilised in discrete spots on a solid support, in indivual wells or containers or on resin beads.

In one embodiment the β-bodies are immobilised on resin beads, e.g. resin beads, useful for on-bead synthesis of β-bodies. Hence, the resin beads may be resins comprising polyethylene glycol, such as PEGA (PolyEthyleneGlycol Acrylamide copolymer; Meldal M., 1992, Tetrahedron Lett., 33: 3077-80), POEPOP

(PolyOxyEthylene-PolyOxyPropylene; Renil et al., 1996, Tetrahedron Lett., 37: 6185-88) or SPOCC (Super Permeable Organic Combinatorial Chemistry; Rademann et al, 1999, J. Am. Chem. Soc., 121: 5459-66). These resins are available in different pore sizes.

In one embodiment of the invention the resin beads are selected from the group consisting of Jandagel® and resin beads comprising polyethylene glycol (PEG). For example, resin beads comprising polyehtylene glycol may be selected from the group consisting of PolyEthyleneGlycol Acrylamide copolymer (PEGA), or PolyOxyEthylene-PolyOxyPropylene (POEPOP), Super Permeable Organic Combinatorial Chemistry (SPOCC), POEPS and Tentagel®.

The library may be a one-bead-one-δ-body library, wherein each bead is linked only to β-bodies of the same sequence. One-bead-one-compound libraries may be prepared according to the principles outlined in Christensen et al., 2003, Lam et al., 1976, or Lam et al., 1991. In short, short libraries may be prepared by a split/mix method comprising the steps of:
1. Providing several pools of resin beads
2. Attaching one amino acid to resin beads of each pool of resin beads, e.g. by SPPS as described above in the section “Method of preparing δ-bodies”, wherein different amino acids may be attached to resin beads of different pools
3. Mixing said pools, thereby obtaining a single pool
4. Splitting said pools to obtain new pools
5. Repeating steps 2 to 4.

The library may be incubated with labelled target compounds and fluorescence intensity of the library beads may be determined and used for selection of β-bodies with desired characteristics. These may be released from the resin and decoded, for example by MS-MS sequencing, or advantageously through micro-particle matrix decoding, which is beneficial in case of modified and/or non-proteinogenic amino acid residues as well as for cyclic peptides. Identified active β-bodies may be resynthesized and binding may be measured to confirm the structure activity relationship.

In one embodiment of the invention, the method of identifying a β-body comprises the steps of
Providing the target compound linked to a detectable label
Providing a one-bead-one-β-body library, e.g. prepared as described above,
Incubating said target compound with said library,
identifying beads associated with the detectable label,
Determining the structure of β-bodies linked to said identified beads.

β-Bodies Binding Target Compound

As mentioned herein above the β-bodies of the invention preferably are capable of binding a target compound with high affinity and/or specificity.

Thus, the β-body may be capable of binding its target compound with a K_{d} of at the most 10⁻⁶ M or less, such as 10⁻⁷ M or less, such as 10⁻⁸ M or less, for example 10⁻⁹ M or less such as 10⁻¹⁰ M, or even 10⁻¹¹M or even less.

Once a β-body that binds to a target compound e.g. a protein has been identified the affinity on said β-body can be improved experimentally by substitution of amino acids X, Z, B or U in said β-body with other similar amino acids. This may be achieved by parallel or focused combinatorial synthesis, e. g. in arrays of columns or by spot synthesis on surfaces.

In one embodiment of the present disclosure the β-body comprises or consists of an amino acid sequence selected from a group consisting of SEQ ID NO:1 to SEQ ID NO: 61.

In one embodiment of the present disclosure the β-body comprises or consists of an amino acid sequence selected from a group consisting of SEQ ID NO:1 to SEQ ID NO: 3, and binds green fluorescent protein (GFP).

In one embodiment of the present disclosure the β-body comprises or consists of an amino acid sequence selected from a group consisting of SEQ ID NO:5 to SEQ ID NO: 13, and binds interleukin 1 (IL1).

In one embodiment of the present disclosure the β-body comprises or consists of an amino acid sequence selected from a group consisting of SEQ ID NO:14 and SEQ ID

NO: 15, and binds interleukin 2 (IL2).

In one embodiment of the present disclosure the β-body comprises or consists of an amino acid sequence selected from a group consisting of SEQ ID NO:16 to SEQ ID NO: 19, and binds interleukin 6 (IL6).

In one embodiment of the present disclosure the β-body comprises or consists of an amino acid sequence selected from a group consisting of SEQ ID NO:20 and SEQ ID NO: 21, and binds interleukin 10 (IL10).

In one embodiment of the present disclosure the β-body comprises or consists of an amino acid sequence selected from a group consisting of SEQ ID NO:22 and SEQ ID NO: 23, and binds interleukin 12 (IL12).

In one embodiment of the present disclosure the β-body comprises or consists of an amino acid sequence selected from a group consisting of SEQ ID NO:24 and SEQ ID NO: 25, and binds interleukin 18 (IL18).

In one embodiment of the present disclosure the β-body comprises or consists of an amino acid sequence selected from a group consisting of SEQ ID NO:26 to SEQ ID NO: 27, and binds tumor necrosis factor alpha (TNFα).

In one embodiment of the present disclosure the β-body comprises or consists of an amino acid sequence selected from a group consisting of SEQ ID NO:28 to SEQ ID

NO: 29, and binds toxin A from Clostridium difficile.

In one embodiment of the present disclosure the β-body comprises or consists of the amino acid sequence SEQ ID NO: 30, and binds botulinum toxin (BTX).

In one embodiment of the present disclosure the β-body comprises or consists of the amino acid sequence SEQ ID NO: 31, and binds ricin.

In one embodiment of the present disclosure the β-body comprises or consists of the amino acid sequence selected from a group consisting of SEQ ID NO:32 to SEQ ID NO: 45, and binds gephyrin.

In one embodiment of the present disclosure the β-body comprises or consists of the amino acid sequence selected from a group consisting of SEQ ID NO:32 and SEQ ID NO: 33, and binds at the protein-protein interface of gephyrin.

In one embodiment of the present disclosure the β-body comprises or consists of the amino acid sequence selected from a group consisting of: SEQ ID NO:34, SEQ ID NO: 41, SEQ ID NO: 42 and SEQ ID NO: 43, and binds the peptide binding site of gephyrin.

In one embodiment of the present disclosure the β-body comprises or consists of the amino acid sequence selected from a group consisting of SEQ ID NO:35 and SEQ ID NO: 36, and binds at the freesite of gephyrin.

In one embodiment of the present disclosure the β-body comprises or consists of the amino acid sequence selected from a group consisting of SEQ ID NO:37 to SEQ ID NO: 40, and binds the molybdenum binding site of gephyrin.

In one embodiment of the present disclosure the β-body comprises or consists of the amino acid sequence selected from a group consisting of SEQ ID NO:46 and SEQ ID NO: 47, and binds subtilisin.

In one embodiment of the present disclosure the β-body comprises or consists of the amino acid sequence selected from a group consisting of SEQ ID NO:48 to SEQ ID NO: 52, and binds papain.

Method of Detection

In one embodiment the invention relates to methods for detecting the presence of a target compound in a sample, said methods comprising the steps of:
a. Providing a sample
b. Providing a β-body, e.g. any of the β-bodies described herein above in the section “β-body”, wherein said β-body is capable of binding said target compound
c. Incubating said sample with said β-body
d. Detecting β-bodies bound to said sample

The β-body may be immobilised on a solid support, e.g. any of the solid supports described herein above in the section “Identifying a β-body”.

In one embodiment the invention relates to methods for detecting the presence of a target compound in a sample, said method comprising
a. Providing a sample
b. Providing at least two different β-bodies, e.g. any of the β-bodies described herein above in the section “β-body”, wherein said β-bodies both are capable of binding said target compound
c. Incubating said sample with said β-bodies
d. Detecting β-bodies bound to said sample

Preferably, said β-bodies are capable of binding to different sites on said target compound. One of said β-bodies may be immobilised on a solid support, and the other β-body may be linked to a detectable label. The step of detecting β-bodies bound to said sample may involve detecting the detectable label associated with the solid support.

In one embodiment the invention relates to methods for detecting the presence of a plurality of target compounds in a sample, said method comprising performing the methods described above for each of the plurality of target compounds.

One of the β-bodies recognising each target compound may be immobilised on individual solid supports, and the other β-body recognising each target compound may be linked to different detectable labels.

The methods for each of the plurality of target compounds may be performed sequentially in either order, partially sequentially or they may be performed simultaneously.

Thus, the b-bodies of the invention may be used for e. g. multiplex diagnostics. Immobilised β-bodies may be used directly in a sandwich assay as a trapping partner for the target compound or the POI.

In one embodiment of the present disclosure the target compound is green fluorescent protein (GFP) and it is recognized by a β-body comprising or consisting of an amino acid sequence selected from a group consisting of SEQ ID NO:1 to SEQ ID NO: 3.

In one embodiment of the present disclosure the target compound is interleukin 1 (IL1) and it is recognized by a β-body comprising or consisting of an amino acid sequence selected from a group consisting of SEQ ID NO:5 to SEQ ID NO: 13.

In one embodiment of the present disclosure the target compound is interleukin 2 (IL2) and it is recognized by a β-body comprising or consisting of an amino acid sequence selected from a group consisting of SEQ ID NO:14 and SEQ ID NO: 15.

In one embodiment of the present disclosure the target compound is interleukin 6 (IL6) and it is recognized by a β-body comprising or consisting of an amino acid sequence selected from a group consisting of SEQ ID NO:16 to SEQ ID NO: 19.

In one embodiment of the present disclosure the target compound is interleukin 10 (IL10) and it is recognized by a β-body comprising or consisting of an amino acid sequence selected from a group consisting of SEQ ID NO:20 and SEQ ID NO: 21.

In one embodiment of the present disclosure the target compound is interleukin 12 (IL12) and it is recognized by a β-body comprising or consisting of an amino acid sequence selected from a group consisting of SEQ ID NO:22 and SEQ ID NO: 23. In one embodiment of the present disclosure the target compound is interleukin 18 (IL18) and it is recognized by a β-body comprising or consisting of an amino acid sequence selected from a group consisting of SEQ ID NO:24 and SEQ ID NO: 25.

In one embodiment of the present disclosure the target compound is tumor necrosis factor alpha (TNFα) and it is recognized by a β-body comprising or consisting of an amino acid sequence selected from a group consisting of SEQ ID NO:26 and SEQ ID NO: 27.

In one embodiment of the present disclosure the target compound is toxin A from Clostridium difficile and it is recognized by a β-body comprising or consisting of an amino acid sequence selected from a group consisting of SEQ ID NO:28 and SEQ ID NO: 29.

In one embodiment of the present disclosure the target compound is botulinum toxin (BTX) and it is recognized by a β-body of an amino acid sequence SEQ ID NO: 30.

In one embodiment of the present disclosure the target compound is ricin and it is recognized by a β-body of an amino acid sequence SEQ ID NO: 31.

In one embodiment of the present disclosure the target compound is gephyrin and it is recognized by a β-body comprising or consisting of an amino acid sequence selected from a group consisting of SEQ ID NO:32 to SEQ ID NO: 45.

In one embodiment of the present disclosure the target compound is subtilisin and it is recognized by a β-body comprising or consisting of an amino acid sequence selected from a group consisting of SEQ ID NO:46 and SEQ ID NO: 47.

In one embodiment of the present disclosure the target compound is papain and it is recognized by a β-body comprising or consisting of an amino acid sequence selected from a group consisting of SEQ ID NO:38 to SEQ ID NO: 52.

Method of Diagnosis

In one embodiment the invention relates to methods for diagnosing a clinical condition, wherein said clinical condition is associated with the presence or absence of one or more target compounds. Such methods may comprise the steps of
a. Providing a sample from an individual at risk of acquiring said clinical condition
b. Performing the method of detecting the target compound(s) described in the section “Method of detection”
c. Wherein the presence or absence of said target compound(s) are indicative of said individual suffering from said clinical condition.

In one embodiment the invention relates to methods for multiplex diagnostics performed with several different β-bodies immobilized on a surface, on a porous material or in a gel matrix. Said materials can for example all be in the form of planar surfaces wells or beads.

Method of Treatment

In one embodiment the invention relates to β-bodies for use in a method of treating a clinical condition, wherein said clinical condition is characterised by expression of a target compound, and wherein said β-body is capable of binding said target compound. The β-body may be any of the β-bodies described herein above in the section “β-body”.

The target compound may be a polypeptide or a protein. The target compound may also be a polysaccharide, oligosaccharide, polypeptide or one or more proteins. Thus, a β-body may for example bridge two proteins in complex.

In one embodiment the β-body may be designed to inhibit a protein protein interaction thereby inhibiting the biological function mediated through said interaction. For example, a β-body of the present disclosure can inhibit the biological functions of an interleukin, such as IL1 , IL2, IL6, IL10, IL12 or IL18. A β-body of the present disclosure can also inhibit the biological functions of TNFα. A β-body of the present disclosure can also inhibit the biological functions of gephyrin, subtilisin or papain. Thus, the β-body may be used in a method of treatment of a clinical condition characterised by increased or undesirable function of any of the aforementioned, in particular immune diseases.

In one embodiment the β-body is for use in a method of neutralizing the toxic effect of is venoms. For example the venom can be toxin A from Clostridium difficile, ricin, or botulinum toxin (BTX). Other venoms can also be targeted by a β-body of the present disclosure.

In one embodiment the β-body is for use in a method of modulating a immune response.

In one embodiment the β-body is for use in a method of modulating apoptosis of cancer cells. Thus, the β-body may be used in a method of treatment of cancer.

In one embodiment the β-body is for use in a method of modulating a hormone hormone receptor response.

Dimers of β-Bodies

The invention also provides dimers of β-bodies, which may be any of the β-bodies described herein above in the section “β-body”. Said dimers may comprise a first β-body and a second β-body, wherein the first and the second β-body are capable of binding each other.

In one embodiment the dimer is a heterodimer comprising a first and a second β-body, wherein said first β-body is different from the second β-body, and wherein said first and second β-bodies are capable of binding each other.

In one embodiment at least 2 amino acids X of first β-body are positively charged, and approximately the same number of amino acids X of the second β-body are negatively charged.

In one embodiment at least 2 amino acids X of first β-body are hydrophobic amino acid residues, and approximately the same number of amino acids X of the second β-body are hydrophobic amino acid residues.

In one embodiment the dimer is a homodimer comprising two identical β-bodies, wherein said β-body is capable of binding to itself.

In one embodiment at least 70%, preferably at least 90% of the amino acids X of said β-body are aromatic or hydrophobic amino acids.

In one embodiment at least 70%, preferably at least 90% of the amino acids X of said β-body are tyrosine residues.

In one embodiment, the invention relates to a dimer comprising a first moiety covalently linked to a first β-body and a second moiety linked to a second β-body, wherein the first and second β-bodies are capable of binding each other. Thus, the first and the second β-body of said dimer may be any of the dimers described in this section above.

In one embodiment said first and/or the second moiety are protein(s).

In one embodiment the first and/or the second moiety are any of the conjugated moieties described herein below in the section “Conjugated moiety”.

In one embodiment said first and the second moiety are different from each other.

In one embodiment said first β-body and said second β-body have a sequence selected from the group consisting of SEQ ID NO: 56; SEQ ID NO: 57; SEQ ID NO: 58; SEQ ID NO: 59; SEQ ID NO: 60.

Conjugated Moiety

The invention also relates to a β-body, e.g. any of the β-bodies described herein above in the section β-body, wherein said β-body is covalently linked to a conjugated moiety.

The conjugated moiety may be any moiety, e.g. it may be selected from the group consisting of detectable labels, such as radiolabels, antigens for antibodies, biotin, fluorescent labels, luminicent labels or colored labels.

The conjugated moiety may also be selected from the group consisting of bioactive compounds such as carbohydrates, polypeptides, proteins, cytotoxic compounds, enzyme inhibitors, enzyme substrates, membrane binding molecules or receptor ligands.

Items
1. A β-body, wherein the β-body is a compound comprising or consisting of at least two β-strand peptide sequences connected by β-turn peptide sequence(s), wherein said β-strand peptide sequences are organized in an anti-parallel arrangement of alternating forward and reverse β-strand peptide sequences, wherein
each forward β-strand peptide sequence individually has the following sequence

Xᵣ(ZX)ₘ

and each reverse β-strand peptide sequence individually has the following sequence

(XZ)ₙXᵣ
wherein

each Z individually is Thr, a polar 13-branched amino acid, non-proteinogenic α-branched amino acids that promote β-strand structure or a strand bridging amino acid, with the exception that at the most two Z in each β-strand sequence may be an amino acid, which is not one of the aforementioned; each X individually is any amino acid, β-amino acid or γ-amino acid; and each m and n individually are integers in the range of 3 to 12; and each r is an integer in the range of 0 to 5; and

and each β-turn peptide sequence individually has the following sequence

X_{q1}BUX_{q2}
wherein
each X individually is any amino acid;

each U individually is an amino acid of the formula

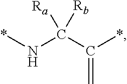

wherein Ra and Rb individually are selected from the group consisting of —H and C₁₋₆-alkyl, wherein Ra and Rb may be linked to form a cyclic structure;

B is selected from the group consisting of Pro, substituted Pro and pipecolic acid;

each q individually is an integer in the range of 0 to 5, wherein q1-q2 is −4, −2, 0, 2 or 4; and

wherein the β-body is linear or cyclic.

2. The β-body according to item 1, wherein the β-body is linear.

3. The β-body according to any one of the preceding items, wherein the compound comprises in the range of 2 to 10 β-strand peptide sequences connected by β-turn peptide sequences.

4. The β-body according to any one of the preceding items, wherein the compound comprises in the range of 2 to 4 β-strand peptide sequences connected by β-turn peptide sequences.

5. The β-body according to any one of the preceding items, wherein said compound have the following structure:

forward β-strand sequence

β-turn peptide sequence

reverse β-strand sequence,

wherein the forward β-strand sequence and the reverse β-strand sequence are arranged as antiparallel β-strands.

6. The β-body according to any one of items 1 to 4, wherein said compound comprises a polypeptide consisting of the following structure:

forward β-strand sequence

β-turn peptide sequence

reverse β-strand sequence

β-turn peptide sequence

forward β-strand sequence

wherein the forward β-strand sequences and the reverse β-strand sequence are arranged as antiparallel β-strands.

7. The β-body according to any one of items 1 to 4, wherein said compound comprises a polypeptide consisting of the following structure:

forward β-strand sequence

β-turn peptide sequence

reverse β-strand sequence

β-turn peptide sequence

forward β-strand sequence

β-turn peptide sequence

forward β-strand sequence.

8. The β-body according to any one of the preceding items, wherein at least 70% of the Z within each β-strand peptide sequences are Thr.

9. The β-body according to any one of the preceding items, wherein at least 90% of the Z within each β-strand peptide sequences are Thr.

10. The β-body according to any one of the preceding items, where at least one forward β-strand sequence has the following sequence

Xᵣ(TX)ₘ

wherein

T is Thr;

each X individually is any amino acid; and

each m individually is an integer in the range of 3 to 12; and

r is an integer in the range of 0 to 5.

11. The β-body according to any one of the preceding items, where at least one reverse β-strand sequence has the following sequence

(XT)ₙXᵣ

wherein

each X individually is any amino acid; and

each n individually is an integer in the range of 3 to 12; and

r is an integer in the range of 0 to 5.

12. The β-body according to any one of the preceding items, wherein at least one β-turn peptide sequence has the following sequence

X_{q}PGX_{q}

wherein

each X individually is any amino acid; and

each q individually is an integer in the range of 0 to 3.

13. The β-body according to anyone of the preceding items, wherein one or more q are integer(s) in the range of 0 to 1. 14. The β-body according to any one of the preceding items, wherein within each β-turn q1-q2 is 0.

15. The β-body according to any one of the preceding items, wherein q1 and q2 individually are integer in the range of 0 to 3.

16. The β-body according to any one of the preceding items, wherein at least one or all β-turn peptide sequences have one of the following sequence

XPGX; or

PGX; or

PG

wherein

each X individually is any amino acid.

17. The β-body according to item 1, wherein the compound comprises a polypeptide having the general structure

(TX)ₘX_{q}PGX_{q}(XT)ₙ,

wherein
each X individually is any amino acid, β-amino acid or γ-amino acid;
m and n individually are integers in the range of 3 to 12; and q individually
are integers in the range of 0 to 3.

18. The β-body according to item 1, wherein the compound comprises a polypeptide having the general structure

(TX)ₘPG(XT)ₙ,

wherein
each X individually is any amino acid, β-amino acid or γ-amino acid; and
m and n individually are integers in the range of 3 to 12.

19. The β-body according to item 1, wherein the compound comprises a polypeptide having the general structure

(TX)ₘPG(XT)ₙXPGX(TX)ₘ

wherein
each X individually is any amino acid, β-amino acid or γ-amino acid; and
each m and n individually are integers in the range of 3 to 12.

20. The β-body according to item 1, wherein the compound comprises a polypeptide having the general structure

(TX)ₘPG(XT)ₙXPGX(TX)ₘPG(XT)ₙ

wherein
each X individually is any amino acid, β-amino acid or γ-amino acid; and
each m and n individually are integers in the range of 3 to 12.

21. The β-body according to item 1, wherein the compound comprises a polypeptide having the general sequence XIX:

Xᵣ(ZX)ₘX_{q}PGX_{q}(XZ)ₙXᵣ,

wherein

each X individually is any amino acid, β-amino acid or γ-amino acid; and

each r individually is an integer in the range of 0 to 5; and
each m and n individually are integers in the range of 3 to 12; and
each Z individually is Thr, a polar β-branched amino acid, non-proteinogenic α-branched amino acids that promote β-strand structure or a strand bridging amino acid, preferably with the proviso that all Z except at the most 2 are Thr;

and each q individually is an integer in the range of 0 to 3.

22. The β-body according to item 1, wherein the compound comprises a polypeptide having the general sequence XVI:

Xᵣ(ZX)ₘPG(XZ)ₙXᵣ,

wherein

each X individually is any amino acid, β-amino acid or γ-amino acid; and

each r individually is an integer in the range of 0 to 5; and
each m and n individually are integers in the range of 3 to 12; and
each Z individually is Thr, a polar β-branched amino acid, non-proteinogenic α-branched amino acids that promote β-strand structure or a strand bridging amino acid, preferably with the proviso that all Z except at the most 2 are Thr.

23. The β-body according to item 1, wherein the compound comprises a polypeptide having the general sequence XXI:

Xᵣ(ZX)ₘX_{q}PGX_{q}(XZ)ₙXX_{q}PGX_{q}X(ZX)ₘXᵣ

wherein
each X individually is any amino acid, β-amino acid or γ-amino acid; and
each r individually is an integer in the range of 0 to 5; and
each m and n individually are integers in the range of 3 to 12; and
each Z individually is Thr, a polar β-branched amino acid, non-proteinogenic α-branched amino acids that promote β-strand structure or a strand bridging amino acid, preferably with the proviso that all Z except at the most 2 are Thr; and
and each q individually is an integer in the range of 0 to 3.

24. The β-body according to item 1, wherein the compound comprises a polypeptide having the general sequence XVII:

Xᵣ(ZX)ₘPG(XZ)ₙXPGX(ZX)ₘXᵣ

wherein
each X individually is any amino acid, β-amino acid or γ-amino acid; and
each r individually is an integer in the range of 0 to 5; and
each m and n individually are integers in the range of 3 to 12; and
each Z individually is Thr, a polar (3-branched amino acid, non-proteinogenic α-branched amino acids that promote β-strand structure or a strand bridging amino acid, preferably the proviso that all Z except at the most 2 are Thr.

25. The β-body according to item 1, wherein the compound comprises a polypeptide having the general sequence XXIII:

Xᵣ(ZX)ₘX_{q}PGX_{q}(XZ)ₙXX_{q}PGX_{q}X(ZX)ₘX_{q}PGX_{q}(XZ)ₙXᵣ

wherein
each X individually is any amino acid, β-amino acid or γ-amino acid; and
each r individually is an integer in the range of 0 to 5; and
each m and n individually are integers in the range of 3 to 12; and
each Z individually is Thr, a polar β-branched amino acid, non-proteinogenic α-branched amino acids that promote β-strand structure or a strand bridging amino acid, preferably with the proviso that all Z except at the most 2 are Thr; and
and each q individually is an integer in the range of 0 to 3.

26. The β-body according to item 1, wherein the compound comprises a polypeptide having the general sequence XVIII:

Xᵣ(ZX)ₘPG(XZ)ₙXPGX(ZX)ₘPG(XZ)ₙXᵣ

wherein
each X individually is any amino acid, β-amino acid or γ-amino acid; and
each r individually is an integer in the range of 0 to 5; and
each m and n individually are integers in the range of 3 to 12; and
each Z individually is Thr, a polar β-branched amino acid, non-proteinogenic α-branched amino acids that promote β-strand structure or a strand bridging amino acid, preferably with the proviso that all Z except at the most 2 are Thr.

27. The β-body according to any one of the preceding items, wherein one or more m are integer in the range of 3 to 7.

28. The β-body according to any one of the preceding items, wherein one or more n are integer in the range of 3 to 5.

29. The 6-body according to any one of the preceding items, wherein one or more, preferably all amino acids X have the general structure

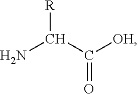

30. The β-body according to any one of the preceding items, wherein one or more amino acids X are selected from the group of substituted glycines of the formula —NH—CHR—CO—, where R may be selected from the group consisting of linear C₁-C₂₀-alkyl, branched C₁-C₂₀-alkyl, aryl, and substituted alkyl.

31. The β-body according to any one of the preceding items, wherein one or more amino acids X are selected from the group of disubstituted glycines of the formula —NH—CR₁R₂—CO—, where R₁ and R₂ individually are selected from the group consisting of linear C₁-C₂₀-alkyl, branched C₁-C₂₀-alkyl, aryl, and substituted alkyl.

32. The β-body according to any one of the preceding items, wherein one or more, preferably all amino acids X are selected from the group consisting of proteinogenic amino acids and non-proteinogenic amino acids, wherein the non-proteinogenic amino acids are selected from the group consisting of α-amino-n-butyric acid, norvaline, norleucine, alloisoleucine, t-leucine, α-amino-n-heptanoic acid, α,β-diaminopropionic acid, α,γ-diaminobutyric acid, ornithine, allothreonine, homocysteine, homoserine, α-aminoisobutyric acid, isovaline, sarcosine, homophenylalanine, propargylglycin and 4-azido-2-aminobutanoic acid.

33. The β-body according to any one of the preceding items, wherein at least some of the amino acid residues of said β-body are L-amino acids.

34. The β-body according to any one of the preceding items, wherein all of the amino acid residues of said β-body are L-amino acids.

35. The β-body according to any one of the preceding items, wherein all of the amino acid residues of said β-body are D-amino acids.

36. The β-body according to any one of the preceding items, wherein at least 70%, such as at least 80%, for example at least 90%, such as all X are standard amino acids.

37. The β-body according to any one of the preceding items, wherein at the most one amino Z, such as none of the amino acids Z are not Thr, a polar β-branched amino acid, non-proteinogenic α-branched amino acids that promote β-strand structure or a strand bridging amino acid. 38. The β-body according to any one of the preceding items, wherein one or more amino acids Z are a β-branched amino acid selected from the group consisting of isoleucine, threonine, allothreonine, alloisoleucine valine, 2-aminoisobutyric acid, 2-amino-3,3-dimethylbutanoic acid, propargylglycin and 4-azido-2-aminobutanoic acid.

39. The β-body according to any one of the preceding items, wherein one or more amino acids Z are a non-proteinogenic α-branched amino selected from the group consisting of α-aminoisobutyric acid, diethylglycine, dipropylglycine, diphenylglycine, 1-aminocyclobutane-1-carboxylic acid, 1-aminocyclopentane-1-carboxylic acid, 1-aminocyclohexane-1-carboxylic acid, and 1-aminocycloheptane-1-carboxylic acid.

40. The β-body according to any one of the preceding items, wherein one or more amino acids Z are strand bridging amino acids selected from the group consisting of cysteine, asparagine, threonine, aspartic acid, glutamic acid, β-amino alanine, γ-amino-α-aminobutyric acid, ornitine, lysine , propargylglycin, 4-azido-2-aminobutanoic acid, amino acids substituted with alkyne, amino acids substituted with azide and amino acids suitable for bridging by reductive amination.

41. The β-body according to any one of the preceding items, wherein each r individually is an integer in the range of 0 to 3, preferably each r is 0.

42. The β-body according to any one of the preceding items, wherein said compound is capable of binding a target compound with a K_{d} of at the most 10⁻⁶ M, for example 10⁻⁷ M or less, such as 10⁻⁸ M or less, such as 10⁻⁹ M or less, for example 10⁻¹⁰ M or less, or even 10⁻¹¹M or even less.

43. The β-body according to any one of the preceding items, wherein said β-body comprises or consists of an amino acid sequence selected from a group consisting of SEQ ID NO:1 to SEQ ID NO: 61.

44. The β-body according to any one of the items 1 to 38, wherein said β-body comprises or consists of an amino acid sequence selected from a group consisting of SEQ ID NO:1 to SEQ ID NO: 3, and wherein said target compound is green fluorescent protein (GFP).

45. The β-body according to any one of the items 1 to 42, wherein said β-body comprises or consists of an amino acid sequence selected from a group consisting of SEQ ID NO:5 to SEQ ID NO: 13, and wherein said target compound is interleukin 1 (IL1).

46. The β-body according to any one of the items 1 to 42, wherein said β-body comprises or consists of an amino acid sequence selected from a group consisting of SEQ ID NO:14 and SEQ ID NO: 15, and wherein said target compound is interleukin 2 (IL2).

47. The β-body according to any one of the items 1 to 42, wherein said β-body comprises or consists of an amino acid sequence selected from a group consisting of SEQ ID NO:16 to SEQ ID NO: 19, and wherein said target compound is interleukin 6 (IL6).

48. The β-body according to any one of the items 1 to 42, wherein said β-body comprises or consists of an amino acid sequence selected from a group consisting of SEQ ID NO:20 and SEQ ID NO: 21, and wherein said target compound is interleukin 10 (IL10).

49. The β-body according to any one of the items 1 to 42, wherein said β-body comprises or consists of an amino acid sequence selected from a group consisting of SEQ ID NO:22 and SEQ ID NO: 23, and wherein said target compound is interleukin 12 (IL12).

50. The β-body according to any one of the items 1 to 42, wherein said β-body comprises or consists of an amino acid sequence selected from a group consisting of SEQ ID NO:24 and SEQ ID NO: 25, and wherein said target compound is interleukin 18 (IL18).

51. The β-body according to any one of the items 1 to 42, wherein said β-body comprises or consists of an amino acid sequence selected from a group consisting of SEQ ID NO:26 to SEQ ID NO: 27, and wherein said target compound is tumor necrosis factor alpha (TNFα).

52. The β-body according to any one of the items 1 to 42, wherein said β-body comprises or consists of an amino acid sequence selected from a group consisting of SEQ ID NO:28 to SEQ ID NO: 29, and wherein said target compound is toxin A from Clostridium difficile.

53. The β-body according to any one of the items 1 to 42, wherein said β-body comprises or consists of the amino acid sequence SEQ ID NO: 30, and wherein said target compound is botulinum toxin (BTX).

54. The β-body according to any one of the items 1 to 42, wherein said β-body comprises or consists of the amino acid sequence SEQ ID NO: 31, and wherein said target compound is ricin.

55. The β-body according to any one of the items 1 to 42, wherein said β-body comprises or consists of the amino acid sequence selected from a group consisting of SEQ ID NO:32 to SEQ ID NO: 46, and wherein said target compound is gephyrin.

56. The β-body according to any one of the items 1 to 42, wherein said β-body comprises or consists of the amino acid sequence selected from a group consisting of SEQ ID NO:47 and SEQ ID NO: 48, and wherein said target compound is subtilisin.

57. The β-body according to any one of the items 1 to 42, wherein said β-body comprises or consists of the amino acid sequence selected from a group consisting of SEQ ID NO:49 to SEQ ID NO: 54, and wherein said target compound is papain.

58. A method for identifying a β-body according to any one of the preceding items, wherein said β-body is capable of binding a target compound, said method comprising the steps of
a. Providing a spatial structure representation of the target compound in a computer;
b. Generating spatial structure representations of a plurality of β-bodies according to any one of the preceding claims in the computer;
c. selecting β-bodies fitting at least part of the spatial structure of the target compound in said computer
thereby identifying a β-body capable of binding the target compound.

59. The method according to item 58, wherein the selected β-body stably associates with the target compound at a temperature in the range of 450 to 200 K for periods in the range of 1 ps-10 s.

60. The method according to any one of items 58 to 57, wherein the method further comprises the following steps
h. Providing a β-body of the spatial structure identified in step c.
i. Providing the target compound
j. Determining whether said β-body is capable of binding said target compound
k. Selecting β-bodies capable of binding said target compound.

61. A method for identifying a β-body according to any one of items 1 to 57, wherein said β-body is capable of binding a target compound, said method comprising the steps of
i. Providing the target compound
ii. Providing library comprising a plurality of test β-bodies according to any one of items 1 to 57
iii. Determining whether said test β-bodies are capable of binding said target compound
iv. Selecting β-bodies most capable of binding said target compound thereby identifying a β-body capable of binding the target compound.

62. The method according to item 61, wherein the library comprises β-bodies immobilised on solid supports.

63. The method according to any one of the items 61 to 62, wherein the library is a one-bead-one-compound library, wherein each bead is linked to β-bodies of the same sequence.

64. The method according to any one of items 61 to 63, wherein the method comprises the steps of
Providing the target compound linked to a detectable label
Providing said one-bead-one-compound library,
Incubating said target compound with said library,
identifying beads associated with the detectable label,
Determining the structure of β-bodies linked to said identified beads.

65. A method for detecting the presence of a target compound in a sample, said method comprising
a. Providing a sample
b. Providing a β-body according to any one of items 1 to 57, wherein said β-body is capable of binding said target compound
c. Incubating said sample with said β-body
d. Detecting β-bodies bound to said sample 66. The method according to item 65, wherein said β-body is immobilised on a solid support.

67. A method for detecting the presence of a target compound in a sample, said method comprising
a. Providing a sample
b. Providing at least two different β-bodies according to any one of items 1 to 57, wherein said β-bodies both are capable of binding said target compound
c. Incubating said sample with said β-bodies
d. Detecting β-bodies bound to said sample 68. The method according to item 67, wherein one of said β-bodies is immobilised on a solid support, and the other β-body is linked to a detectable label.

69. The method according to item 64, wherein the step of detecting β-bodies bound to said sample involves detecting the detectable label associated with the solid support.

70. A method for detecting the presence of a plurality of target compounds in a sample, said method comprising performing the method according to any one of items 65 to 69 for each of the plurality of target compounds.

71. The method according to item 70, wherein one β-body recognising each target compound is immobilised on individual solid supports, and the other β-body recognising each target compound are linked to different detectable labels.

72. The method according to item 71, wherein the method according to any one of items 65 to 69 for each of the plurality of target compounds are performed simultaneously.

73. A method for detection of interaction between β-bodies and target compounds, said method comprising immobilizing a plurality of β-bodies on a solid surface in a microarray format, and contacting said microassay with one or more target compound(s) linked to a detectable label, thereby detecting interaction with target compounds.

74. A method for neutralizing the toxic effect of a venom, said method comprising
a. Providing a sample comprising a venom
b. Providing a β-body according to any one of items 1 to 57, wherein said β-body is capable of binding said toxin
c. Incubating said sample with said β-body.

75. A method for treating a clinical condition, wherein said clinical condition is associated with the presence of a venom, said method comprising the steps of:
a. providing a sample from an individual affected by or suspected of being affected by said clinical condition
b. performing the method of neutralizing a toxin according to item 74.

76. A method for diagnosing a clinical condition, wherein said clinical condition is associated with the presence or absence of one or more target compounds, said method comprising the steps of
a. Providing a sample from an individual at risk of acquiring said clinical condition
b. Performing the method of detecting the target compound(s) according to any one of items 65 to 72
c. Wherein the presence or absence of said target compound(s) are indicative of said individual suffering from said clinical condition.

77. A β-body according to any one of items 1 to 57 for use in a method of treating a clinical condition, wherein said clinical condition is characterised by expression of a target compound, and wherein said β-body is capable of binding said target compound.

78. The β-body according to item 77, or the method according to any one of items 58 to 71, wherein the target compound is a polypeptide.

79. A heterodimer comprising a first and a second β-body according to any one of items 1 to 57, wherein said first β-body is different from the second β-body, and wherein said first and second β-bodies are capable of binding each other.

80. The heterodimer according to item 79, wherein at least 2 Xs of first β-body are positively charged, and approximately the same amount of Xs of the second β-body are negatively charged.

81. The heterodimer according to any one of items 79 to 80, wherein at least 2 Xs of first β-body are hydrophobic amino acid residues, and approximately the same amount of Xs of the second β-body are hydrophobic amino acid residues.

82. A homodimer comprising two identical β-bodies, wherein the each β-body is according to any one of items 1 to 57 wherein said β-body is capable of binding to itself.

83. The homodimer according to item 82, wherein at least 70%, preferably at least 90% of the Xs of said β-body are aromatic or hydrophobic residues.

84. The homodimer according to any one of items 82 to 83, wherein at least 70%, preferably at least 90% of the Xs of said β-body are tyrosine residues.

85. A dimer comprising a first moiety covalently linked to a first β-body and a second moiety linked to a second β-body, wherein the first and the second β-body are β-bodies according to any one of items 1 to 57, and wherein the first and second β-bodies are capable of binding each other.

86. The dimer according to item 85, wherein the first and the second β-bodies are capable of forming a heterodimer according to any one of items 79 to 81.

87. The dimer according to item 86, wherein the first and the second β-bodies are capable of forming a homodimer according to any one of items 82 to 84. 88. The dimer according to any one of items 85 to 87, wherein the first and/or the second moiety are protein(s).

89. The dimer according to any one of items 85 to 88, wherein the first and the second moiety are different from each other.

90. Use of one or more β-bodies, wherein the each β-body is according to any one of items 1 to 57 in a method of affinity chromatography or as a fusion partner of a protein.

91. A compound comprising the β-body according to any one of items 1 to 57, wherein said β-body is covalently linked to a conjugated moiety.

92. The compound according to item 91, wherein the conjugated moiety is selected from the group consisting of detectable labels, such as radiolabels, antigens for antibodies, biotin, fluorescent labels, luminescent labels or colored labels.

93. The compound according to item 91, wherein the conjugated moiety is selected from the group consisting of bioactive compounds such as polypeptides, proteins, cytotoxic compounds, enzyme inhibitors, enzyme substrates, membrane binding molecules or receptor ligands.

94. The compound according to item 91, wherein the conjugated moiety is a polypeptide.

95. The β-body according to any one of items 1 to 57, the heterodimer, homodimer or dimer according to any one of items 79 to 89 or the compound according to any one of items 91 to 94, wherein the β-body is designed to inhibit a protein-protein interaction thereby inhibiting the biological function mediated through said interaction.

96. The β-body according to any one of items 1 to 57, the heterodimer, homodimer or dimer according to any one of items 79 to 89 or the compound according to any one of items 91 to 94, wherein the β-body is for use in a method of neutralizing the toxic effect of venoms.

97. The β-body according to any one of items 1 to 57, the heterodimer, homodimer or dimer according to any one of items 79 to 89 or the compound according to any one of items 91 to 94, wherein the β-body is for use in a method of modulating an immune response.

98. The β-body according to any one of items 1 to 57, the heterodimer, homodimer or dimer according to any one of items 79 to 89 or the compound according to any one of items 91 to 94, wherein the β-body is for use in a method of modulating a hormone hormone receptor response.

### EXAMPLES

### Example 1

Design of β-bodies against known protein structures.

The design of β-bodies specifically binding known protein structures started with a search for the protein of interest (POI) in e.g. PDB

(http://www.rcsb.org/pdb/home/home.do). The protein structure was acquired as the PDB file. All modelling was performed with Molecular Operating Environment (MOE—ver.2015.10, and the force fields ETH10 or ETH12) from Chemical Computing Group. The PDB-file was loaded, hydrogen atoms were added and the structure thoroughly investigated and corrected for any missing parts e. g. by homology modelling or by restrained dynamics if possible. The model was fixed in space and was equipped with a molecular electrostatic surface.

A spatial structure of two-stranded β-bodies of the structure (TX)ₘPG(XT)ₙ, in which the PG constitute a type 2 β-turn flanked by two threonine rich β-strands and in which X was initially alanine was constructed. Up to 30% of the threonines can be randomly replaced either with other β-branched or strand bridging amino acids when required for molecular interaction with the POI, but generally the threonine side of the strand faces the solvent during protein binding. The turn region could also be modified to constitute other sequences as found in naturally occurring β-turns in protein crystal structures or with unnatural amino acid sequences known to induce β-turns.

The spatial structure of the initial T/A rich β-body was manually moved and rotated across the entire surface of the POI to identify the sites for optimal interaction in terms of overall shape fitting and presence of grooves, pits and patches promising for interaction with amino acid side-chains. The one to three most promising orientations of the T/A rich β-body was selected for alanine side-chain replacement to fit each side-chain optimally into the selected binding side. During this process many rounds of fitting using molecular dynamics by annealing (450-300 K, step 0.5 fs) with 8-10 layers of added layers of water was performed to take into account the additive effects of amino acid side-chain orientation, H-bond network, hydrophobic interaction, charge-charge interaction. When the rough model was obtained the amino acids in the POI in direct contact with the β-body were released from fixation while the rest of the POI structure remained fixed. Refinement of the interaction and final adjustment of side-chains was performed. The entire optimization was followed by assessing the electrostatic interaction of the two surfaces on the POI and β-body, respectively, to eventually obtain the maximum overlap of positive with negative and hydrophobic with hydrophobic patches on the surface. Most importantly the size (molecular space) of the side-chains of the β-body should allow the maximum uninterrupted overlap of the surfaces between POI and β-body, thereby providing a protein—β-body interaction complementarity that excluded water molecules optimally. The final β-body—POI—water complex was first allowed to relax (with most of the POI still fixed) during dynamics calculations at 300 K for 1-2 ns.

The predetermined 3-dimentional structure of the β-body is of great importance for the affinity obtained. Therefore the POI was removed and the β-body was immerged in a wall-constrained droplet of water. Molecular dynamics was continued at 300 K for 1-2 ns to access the structural stability and integrity of the β-body. If this for some reason was not stable the procedure was looped from anywhere in the above until structural stability could be obtained give sufficient stability. During this process it would often make sense to use either constraining pairs on the threonine side of the β-body, such as clickable acido- alkyne amino acids or disulfide bonds. It would also help to use the β-branched isoleucine or valine at hydrophobic patches in the interaction, even at the expense of less optimal surface fitting.

TABLE 1
List of β-bodies that were designed. Those β-bodies that were tested for
binding with the target compound, and binding was achieved, are marked with a (^{T})

| | | | SEQ | |
|---|---|---|---|---|
| Purpose | Structure | Context | ID NO: | Binding |
| eGFP | TETKTVTITRPKMTWTFTHTVTG(^{T}) | | 1 | High |
| eGFP | Pra-ETKTVTITRPKMTWTFTHTV-Abu(N₃)-G-OH | Cyclized | 2 | |
| EGFP- | KTGTQNLTGPGRTHTQTATEG | | 3 | |
| Ex. 5 | | | | |
| Ex. 5 | HRMVRG | | 4 | |
| Il1 | ETDTYTETYPGYTSTWTITD(^{T}) | bead | 5 | Very |
| | | | | high |
| Il1 | TWTDTATEPGYTMTATGT | RoxNH | 6 | |
| Il1 | TKTDRVTEPGRTMTFTGT | RoxNH | 7 | |
| Il1 | Pra-ETDTYTETYPGYTSTWTITD(^{T}) | bead | 8 | Very |
| | | | | high |
| Il1 | EPraDTYTETYPGRTITWTIAbu(N₃)DG(^{T}) | Bead | 9 | High |
| | | cyclized | | |
| Il1 | GE Abu(N₃)ITSTVTDPGKTDTVQNPraG | RoxNH | 10 | |
| | | cyclized | | |
| Il1 | TWTETYTWTEPGDTQTLTITNT(^{T}) | binder | 11 | High |
| Il1 | TWTKTGTAPGLTVRYTYT | binder | 12 | |
| Il1 | ETYTETYPGYTSTWTIDD | binder | 13 | |
| Il2 | TRTLTYTEPGITQTKTEA(^{T}) | Bead | 14 | High |
| Il2 | NTVTNTMTRPGVTETVTQTD(^{T}) | RoxNH | 15 | High |
| Il6 | TMTDTDTYPGFTDTLTHA(^{T}) | Bead | 16 | Very |
| | | | | high |
| Il6 | HTWTDTLTRPGYTVTHTLTL(^{T}) | RoxNH | 17 | Very |
| | | | | high |
| Il6 | GPraSTWTMTNPGWTKTHTLAbu(N₃)G | Bead | 18 | |
| | | cyclized | | |
| Il6 | GGHAbu(N₃)WTDTLTRPGYTVTHTLPraLG | RoxNH | 19 | |
| | | cyclized | | |
| Il10 | ATNTLTMTWPGRTNTDTFTW(^{T}) | Bead | 20 | High |
| Il10 | TKTRTYTIPGERYTDTWA(^{T}) | RoxNH | 21 | High |
| Il12 | TLTFTATRPGLTKTITITL | Bead | 22 | |
| Il12 | DTVTKTFTWPGAKLTFTKT | RoxNH | 23 | |
| Il18 | KTWTLTHTKPGNTATDTHTI | Bead | 24 | |
| Il18 | RLTWTMTIPGLTLTLTDT | RoxNH | 25 | |
| TNFa | TWTLTWTKPGQEQTMTHA | Bead | 26 | |
| TNFa | YTLTDTETYPGHTRTATQTE | RoxNH | 27 | |
| Clostridium | wEHTHTeTSPGNTQTST | Two D | 28 | |
| difficile | amino | | | |
| acids | | | | |
| Clostridium | wEHTHTeTSPGNTYTST | Best | 29 | |
| difficile | | | | |
| Botulinum | E-Abu(N₃)FTMEQTWTGPGSTKTFTFTH-Pra-G | cyclized | 30 | |
| toxin | | | | |
| Ricin | LTFTFTVTPGTFTWTGTKPGETYTFTRTE | Sheet | 31 | |
| Gephyrin | Pra-KTKTWTMTGPGGEKTRTLTA-Abu(N₃)-G- | Cyclized | 32 | |
| | OH | | | |
| Gephyrin | Pra-WTNTGTYTIPGVTVTMTETV-Abu(N₃)-E | Cyclized | 33 | |
| Gephyrin | Pra-TVTGTLYPGTLLGFET-Abu(N₃)(^{T}) | Cyclized | 34 | Very |
| | | | | high |
| Gephyrin | Ac-VTWTDTLTFTLPGVTWTITMTITE | freesite | 35 | |
| Gephyrin | HTLTKTITQTWPGKTYTITWTFTW | freesite | 36 | |
| Gephyrin | KTW-Pra-LTITPGTMEI-Abu(N₃)-DTV | Cyclized | 37 | |
| Gephyrin | Pra-YTYTDTTPGVTRTLTWG-Abu(N₃)-OH(^{T}) | Cyclized | 38 | Medium |
| Gephyrin | PraTWTLTHTPGTMEITET-Abu(N₃)-OH(^{T}) | Cyclized | 39 | Medium |
| Gephyrin | TW(6-NH₂)TLTHTPGTMEITET-Abu(N₃)-OH | Cyclized | 40 | |
| Gephyrin | TTVTGTLYPGTLLGFETT(^{T}) | | 41 | Very |
| | | | | high |
| Gephyrin | CTVTGTLYPGTLLGFETC(^{T}) | | 42 | Very |
| | | | | high |
| Gephyrin | TTVTGTLYPGTLLGAATT(^{T}) | | 43 | Very |
| | | | | high |
| Gephyrin | Abu(N₃)-TVTGTLYPGTLLGFET-Pra | | 44 | |
| Gephyrin | TITKTARYTMPGKTLTKTGTLTG(^{T}) | | 45 | Medium |
| Gephyrin | Pra-ITKTARYTMPGKTLTKTGTL-Abu(N₃)-G(^{T}) | Cyclized | 46 | Medium |
| Subtilisin | tltmtwtythtpGtitwtytdtttG-OH | D-amino | 47 | |
| | | acids | | |
| Subtilisin | abu(N₃)-ltmtwtythtpGtitwtytdtt-pra-G-OH | D-amino | 48 | |
| | | acids | | |
| | | Cyclized | | |
| Papain | IHV-abu(N₃)-VTTRTMPGHPIAGADA-pra-TG(^{T}) | twisted & | 49 | Medium |
| | | cyclized | | |
| Papain | tqtmtGtltwtpGtqtntltwtftG | D-amino | 50 | |
| | | acids | | |
| Papain | abu(N₃)-qtmtGtltwtpGtqtntltwtf-pra-G | D-amino | 51 | |
| | | acids | | |
| | | Cyclized | | |
| Papain | abu(N₃)-wtftlpqGtmtn-pra-G | D-amino | 52 | |
| | | acids | | |
| | | Cyclized | | |
| Papain | etltwtgtvtvtfpGitmtttEtmtftf-OH | D-amino | 53 | |
| | | acids | | |
| Papain | abu(N₃)-wtvtvtfpGitmtttf-pra-OH | D-amino | 54 | |
| | | acids | | |
| | | Cyclized | | |
| Ex. 4 | KTQTYNGTGPGRTGTVTYTEG | | 55 | |
| Ex. 4 | KTYTYNYTGPGRTSTATLTEG | | 56 | |
| Heterodimer- | TYTYTYPGLTRTHT | | 57 | |
| Ex. | | | | |
| 7 | | | | |
| Heterodimer- | TTYTYPGDTFTI | | 58 | |
| Ex. | | | | |
| 7 | | | | |
| Heterodimer- | TFTFTFPGLTRTHT | | 59 | |
| Ex. | | | | |
| 7 | | | | |
| Heterodimer- | TDTRTYTYTVPGRTRTRTWTET | | 60 | |
| Ex. | | | | |
| 7 | | | | |
| Heterodimer- | DTITYTYTGPGRTDTETNTEG | | 61 | |
| Ex. | | | | |
| 7 | | | | |

### Example 2

### Sandwich Assay

β-bodies may be used for Sandwich assays, where two different β-bodies binds the same POI at different sites.

For the Sandwich assay of two β-bodies with the POI the procedure described in Example 1 was repeated for the second or third best site on the POI identified with the T/A—β-body above and at the same time securing no unwanted specific interaction between the two β-bodies would occur. This was done by ensuring surface mismatching in the two molecules during restrained (maintaining the β-body backbone structures) molecular dynamics at 300 K of the pair in a water droplet.

### Example 3

### Sandwich Assay

For sandwich assays β-bodies identified as described in Examples 1 and 2 were synthesized by standard Fmoc-based solid phase peptide synthesis on biocompatible PEGA-beads (e.g. PEGA₁₉₀₀ which has a porosity allowing penetration of proteins up to 70 kDa). PEGA beads are available from Sigma Aldrich.

In an example of a Sandwich assay the crystal structures of interleukins (IL1, IL2, IL6, IL10, IL12, IL18 and TNFα) are subjected to the procedure described in Examples 1 and 2.

IL1 Model

For IL1 two β-bodies binding to opposite sides of IL1 were identified as described in Example 1 and 2 and synthesized as by solid phase as follows:

A: Ligand 1: (SEQ ID NO: 5) ETDTYTETYPGYTSTWTITD—Bead (synthesized, deprotected and used while still attached to the _{PEGA1900} resin. A small fraction was released from the hydroxymethylbenzamide (HMBA) linker used and characterized by HRMS)

B: Ligand 2: RhodamineX—(SEQ ID NO: 7) TKTDRVTEPGRTMTFTGT-OH

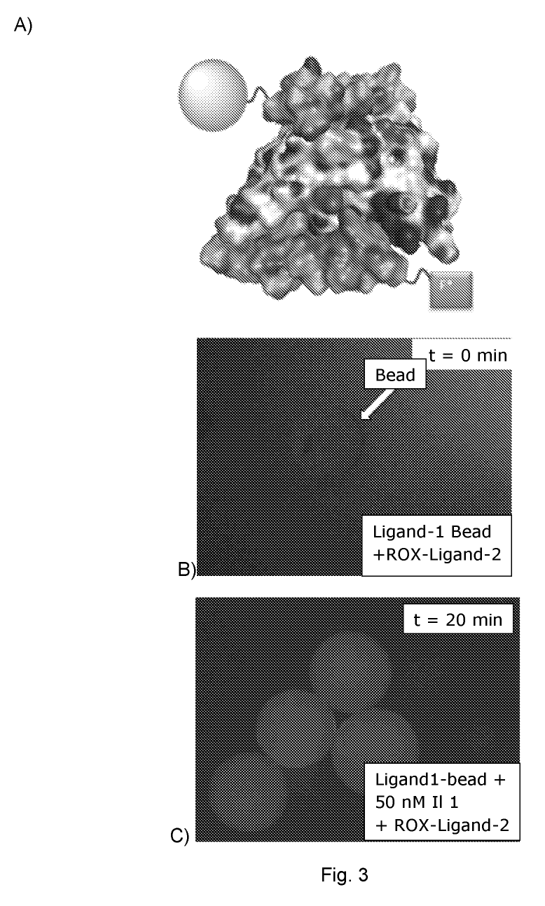

(Synthesized on HMBA-PEGA₈₀₀ released by treatment with 0.1 M NaOH, purified by preparative HPLC, lyophilized and characterized by HRMS.) A model of IL-1 bound to ligand 1 and ligand 2 is shown in FIG. 3A.

To demonstrate the sandwich binding assay four beads of the peptide A above were added to 50 μL Milli-Q water in a microtiter well containing 100 nM of the peptide B. The well was imaged with an ICX73 fluorescence microscope (Olympus) using a ROX filter cube. No fluorescence accumulation over background could be detected (see FIG. 3B). This indicated that there was no specific binding interaction between the two β-bodies. To this was added a solution of Interleukin 1 (50 nM) and after a short period of time the accumulation of significant ROX fluorescence in the beads was observed as an indication that the IL1 bound to A and recruited B to the beads. Fluorescence after 20 min. incubation is shown in FIG. 3C. The intensity of the fluorescence is a measure of the concentration of IL1 and the affinity of the interaction.

IL2 Model

For IL2 two β-bodies binding to opposite sides of IL2 were identified as described in Examples 1 and 2 and synthesized as follows:

C: Ligand 3: NTVTNTMTRPGVTETVTQTD (SEQ ID NO: 15) was synthesized by solid phase synthesis directly on PEGA1900 resin beads. The ligand was attached to the bead via a hydroxymethylbenzamide (HMBA) linker. The ligand was synthesized, deprotected and used while still attached to the PEGA₁₉₀₀ resin. A small fraction was released from the hydroxymethylbenzamide (HMBA) linker used and characterized by HRMS

D: Ligand 4: TRTLTYTEPGITQTKTEA (SEQ ID NO: 14) linked to the fluorophore RhodamineX. (Ligand 4 was synthesized on HMBA-PEGA₈₀₀ beads and released by treatment with o.1 M NaOH, purified by preparative HPLC, lyophilized and characterized by HRMS.

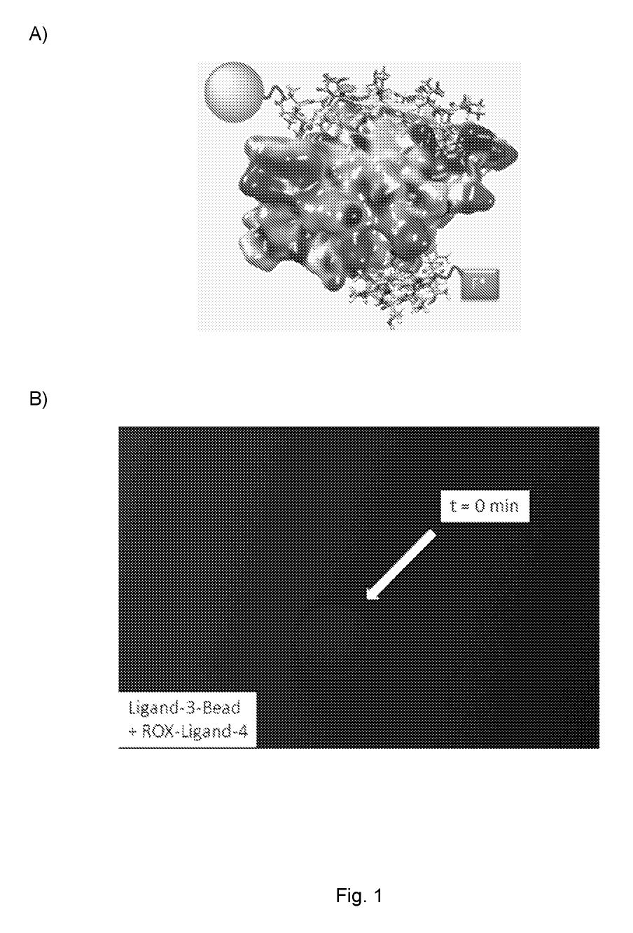

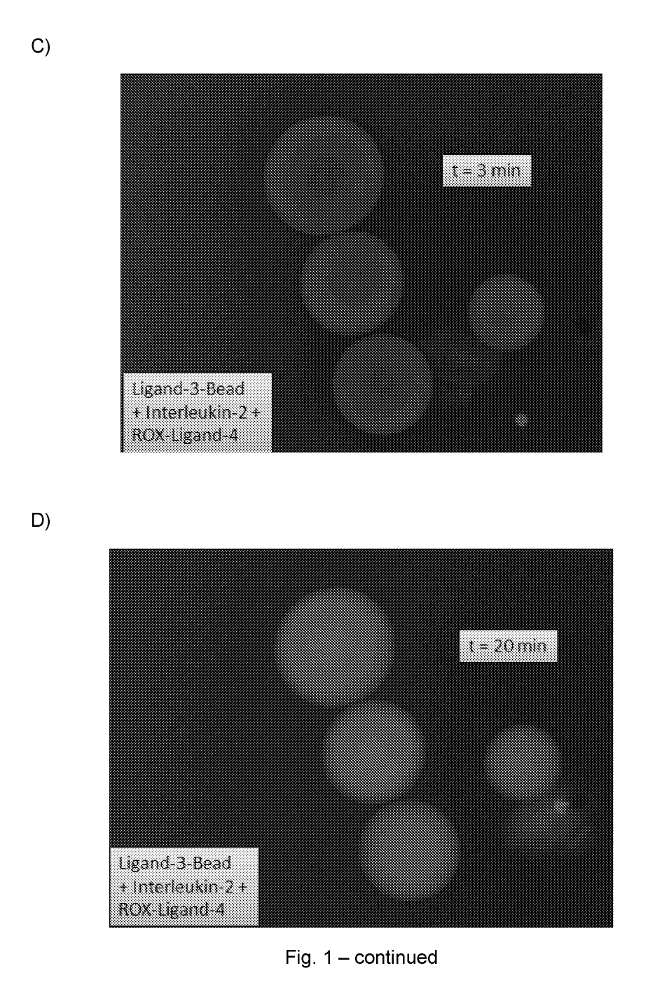

A model of IL-2 bound to ligand 3 and ligand 4 is shown in FIG. 1A.

The sandwich binding assay was performed as follows: four beads with ligand C prepared as described above were added to 50 μL MilliQ water in a microtiter well containing 100 nM of the peptide D. The well was imaged with an ICX73 fluorescence microscope (Olympus) using a ROX filter cube. No fluorescence accumulation over background could be detected (see FIG. 1B). This indicated that there was no specific binding interaction between the two β-bodies.

To this was added a solution of Interleukin 2 (50 nM) and after a short period of time the accumulation of significant ROX fluorescence in the beads was observed as an indication that the IL2 bound to C and recruited D to the beads. The intensity of the fluorescence is a measure of the concentration of IL2 and the affinity of the interaction. The result obtained after 3 min. incubation is shown in FIG. 10 and after 20 min. incubation in FIG. 1D.

IL6 Model

For IL6 two β-bodies binding to opposite sides of IL6 were identified as described in Examples 1 and 2 and synthesized as follows:

E: Ligand 5: HTWTDTLTRPGYTVTHTLTL (SEQ ID NO: 17) linked to PEGA₁₉₀₀-beads was synthesized by solid phase synthesis directly on PEGA₁₉₀₀ resin beads. The ligand was attached to the bead via a hydroxymethylbenzamide (HMBA) linker. The ligand was synthesized, deprotected and used while still attached to the PEGA₁₉₀₀ resin. A small fraction was released from the hydroxymethylbenzamide (H MBA) linker used and characterized by HRMS

F: Ligand 6: TMTDTDTYPGFTDTLTHA (SEQ ID NO: 16) linked to the fluorophore RhodamineX. (Ligand 6 was synthesized on HMBA-PEGA₈₀₀ beads and released by treatment with 0.1 M NaOH, purified by preparative HPLC, lyophilized and characterized by HRMS.

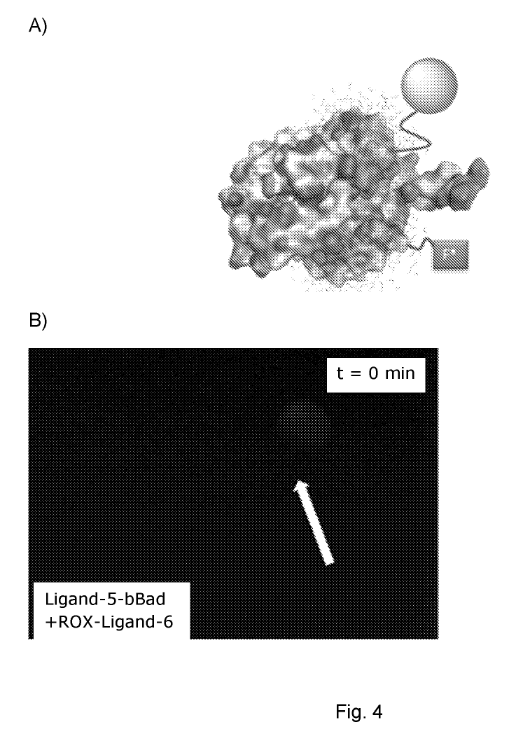

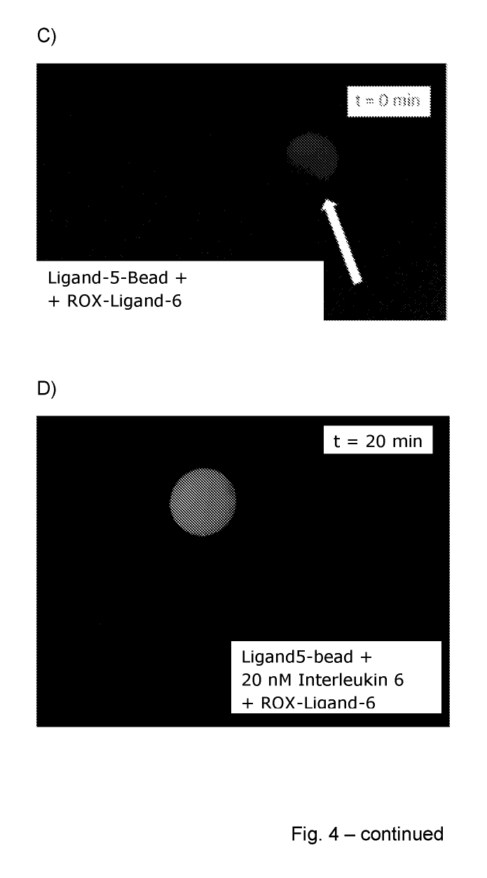

A model of IL-6 bound to ligand 5 and ligand 6 is shown in FIG. 4A.

The sandwich binding assay was performed as follows: two beads with ligand E prepared as described above were contained in separate wells and were added to 50 μL MilliQ water in a microtiter well containing 100 nM of the peptide F. The mixtures were incubated for several hours.

To one of the wells was added a solution of interleukin 6 (20 nM) and after a short period of time the accumulation of significant ROX fluorescence in the bead was observed in the well with interleukin 6 and not in the other well as an indication that the IL6 bound to E and recruited F to the bead while this did not happen in absence of interleukin 6. The beads were washed twice with PBS to improve signal to noise. The well was imaged with an ICX73 fluorescence microscope (Olympus) using a ROX filter cube. No fluorescence accumulation over background (see FIG. 4B and enhanced image 4C) could be detected in the well without IL 6 while strong fluorescence was observed in presence of IL 6. This indicated that there was no specific binding interaction between the two β-bodies while the intensity of the fluorescence was a measure of the concentration of IL6 and the affinity of the interaction. The result obtained after 20 min. incubation with 20 nM IL6 is presented in FIG. 4D.

### Example 4

Selection of 13-bodies from molecular libraries of β-bodies.

Selection of binding partners for molecular interaction by combinatorial methods is a very powerful technique for identification of high affinity molecules for a particular interaction. Phage display libraries with point and site mutation of larger protein structures has been used extensively for finding proteins that bind to POI's. However these are larger molecules only accessible by protein expression. The present invention may use combinatorial synthesis and screening of Solid Phase One Bead One Compound (OBOC) libraries of β-bodies to identify compounds of high affinity to a POI.

The library of K(TX)₄PG(XT)₄EG was synthesized on HMBA-PEGA₁₉₀₀ resin by the split—mix method essentially as described in Christensen et al, 2003 and was deprotected with 95% TFA. The POI was dissolved in phosphate buffer at pH 7.5, labeled with aminomethylcoumarin using AMC-(CH₂)₃—CO—OSu (4 eqv, 30 min) and purified by FPLC. HRMS indicated that the POI contained 1 AMC group. The protein was dissolved at 100 nM concentration and the library was incubated for 2 h with this solution at 10 fold dilution (10 nM). The library was inspected under a ICX73 fluorescence microscope using a GFP filtercube. Beads with strong AMC-fluorescence were collected in separate Eppendorph tubes using a microsyringe. The beads were washed and 5% triethylamine in water was added. After incubation overnight the supernatant was transferred to another eppendorph tube and the bead was washed with 70% acetonitril/water. The combined solutions were concentrated to dryness twice using a speedvacc and the residue dissolved in 50% acetonitrile/water. The product was spottet on a MALDI plate with a-cyano-4-hydroxy cinnamic acid matrix and analyzed by MSMS sequencing on a Bruker Solarix ICR-instrument providing strong binding β-body sequences such as KTQTYNGTGPGRTGTVTYTEG (SEQ ID NO: 55), KTYTYNYTGPGRTSTATLTEG (SEQ ID NO: 56) and KTGTQNLTGPGRTHTQTATEG (SEQ ID NO: 3). The site of interaction on the POI was not determined.

### Example 5

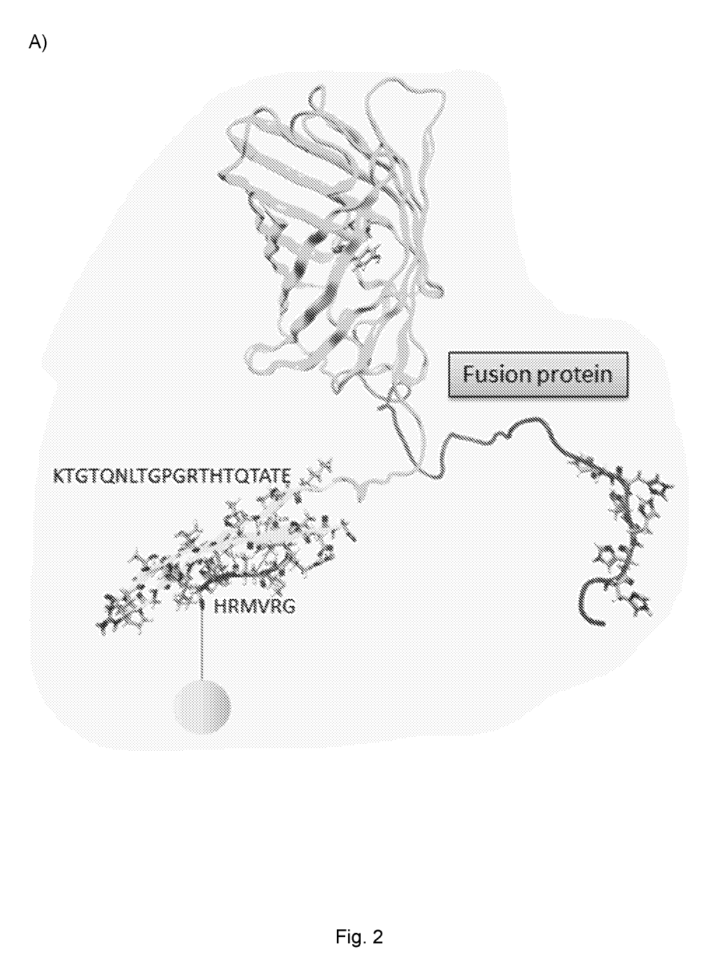

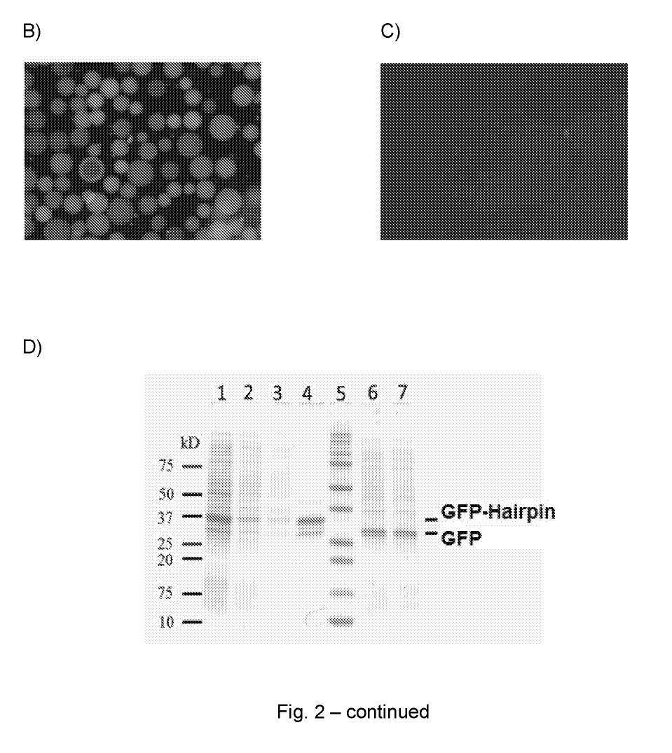

This example illustrates how to identify a β-body recognizing a linear peptide. A split-mix library of 21.000.000 β-bodies according to the invention were prepared by combinatorial synthesis as described in Example 4 above. This was performed on 100 μm beads with no fluorescence. A second split mix library of 470.000 hexapeptides were prepared in a similar manner except that this library was prepared on larger 400 μm beads with fluorescence label attached to some functional groups. The two libraries were mixed and pairs of beads adhering to each other in pairs with one large fluorescent and one small non-fluorescent bead were collected and the two peptides were identified by MSMS. Resynthesis of the peptides and bead binding assays showed high specificity of pair interaction and binding con-stants K_{d} from 10⁻⁸-10⁻⁶ M. The β-body peptide pair (KTGTQNLTGPGRTHTQTATEG (SEQ ID NO: 3) and HRMVRG (SEQ ID NO: 45)) was used to prepare an EGFP fusion protein containing: histag—EGFP—Spacer—KTGTQNLTGPGRTHTQTATEG (SEQ ID NO: 3). A model of this fusion protein bound to the hexapeptide linked to the bead is shown in FIG. 2A. EGFP is an abbreviation of Enhanced Green Fluorescent Protein.

The fusion protein as well as EGFP was overexpressed in E. coli and purified from the cell lysate using a PEGA solid support with the hexapeptide partner, HRMVRG (SEQ ID NO: 4), attached. The EGFP-β-body fusion protein binds to (SEQ ID NO: 5) HRMVRG-PEGA₁₉₀₀ as evidenced by green fluorescence associated with the beads (see FIG. 2B), whereas EGFP does not (see FIG. 2C).

For purification, the fusion protein was overexpressed in E. coli and the cells were lyzed. The cell lysate was centrifuged and the supernatant was passed through an affinity column containing (SEQ ID NO: 5) HRMVRG-PEGA₁₉₀₀. The column was washed with water and the protein was eluted with PBS buffer at pH 6. The purified EGFP-fusion protein was eluted with PBS and was pure according to FPLC and MS.

FIG. 2D shows an SDS-PAGE analysis of various fractions obtained during the purification. Column 4 shows the eluate, whereas column 1 shown the crude extract. The expected size of the fusion protein is indicated as “GFP-hairpin”, whereas the expected size of EGFP is indicated as “GFP”.

Affinity Purification of EGFP

EGFP expressed in E. coli; cells were spun down and lysed. The lysate, both untreated (EGFP 150 μM) and diluted (EGFP 500nM) was incubated with β-body beads (β-body SEQ ID NO: 1; TETKTVTITRPKMTWTFTHTVTG).

FIG. 6 shows the EGFP-β-body fusion complex both in the untreated (A) and diluted (B) lysate.

This example illustrates how β-bodies can be used in affinity purifications.

### Example 6

### Selectivity Assay: GFP vs. IL1

GFP (60 nM) was incubated with different β-body beads at the same time, a first type of β-body beads specific for GFP (β-body SEQ ID NO: 1;

TETKTVTITRPKMTWTFTHTVTG) and a second type of β-body beads specific for IL1 (β-body SEQ ID NO: 5).

FIG. 7A shows that only β-body beads specific for GFP underwent binding.

The same experiment was repeated with IL1. ROX-IL1 (100 nM) was incubated with different β-body beads at the same time, a first type of β-body beads specific for GFP (β-body SEQ ID NO: 1) and a second type of 13-body beads specific for IL1 (β-body SEQ ID NO: 5; ETDTYTETYPGYTSTWTITD).

FIG. 7B shows that only β-body beads specific for IL1 underwent binding.

### Example 7

### β-Bodies for Inhibition of Gephyrin

Gephyrin is a 93 kDa multifunctional protein consisting of 3 domains: N-terminal G domain, C-terminal E domain and an unstructured linker domain connecting the T-terminal with the terminal domains. In cells, gephyrin appears to form oligomers of at least 3 subunits. Several β-bodies targeting different sites of gephyrin have been designed.

In particular:

a) two β-bodies targeting the protein-protein interface:

(SEQ ID NO: 32)
Pra-KTKTWTMTGPGGEKTRTLTA-Abu(N₃)-G-OH,
and
(SEQ ID NO: 33)
Pra-WTNTGTYTIPGVTVTMTETV-Abu(N₃)-E;

b) β-body targeting the peptide binding site:

(SEQ ID NO: 34)
Pra-TVTGTLYPGTLLGFET-Abu(N₃);
(SEQ ID NO: 41)
TTVTGTLYPGTLLGFETT;
(SEQ ID NO: 42)
CTVTGTLYPGTLLGFETC;
(SEQ ID NO: 43)
TTVTGTLYPGTLLGAATT;
and
(SEQ ID NO: 44)
(Ábu(N₃)-TVTGTLYPGTLLGFET-Pra;

c) four β-bodies targeting the molybdenum binding site:

(SEQ ID NO: 39)
PraTWTLTHTPGTMEITET-Abu(N₃)-OH, inverted,,
(SEQ ID NO: 40)
PraTW(6-NH₂)TLTHTPGTMEITET-Abu(N₃)-OH, inverted,,
(SEQ ID NO: 38)
Pra-YTYTDTTPGVTRTLTWG-Abu(N₃)-OH, normal,,
and
(SEQ ID NO: 37)
KTW-Pra-LTITPGTMEI-Abu(N₃)-DTV, optimized not b-hp,;

d) freesite:

(SEQ ID NO: 36)
HTLTKTITQTWPGKTYTITWTFTW,
and
(SEQ ID NO: 35)
Ac-VTWTDTLTFTLPGVTWTITMTITE.

The following β-body TTVTGTLYPGTLLGFETT (SEQ ID NO: 41) underwent optimization via alanine scanning. The resulting 9 β-bodies were tested for their affinity to the glycine receptor binding site of gephyrin (peptide binding site). The affinity of the original peptide could be improved and optimized β-bodies were found having the following sequences CTVTGTLYPGTLLGFETC (SEQ ID NO: 42) and TTVTGTLYPGTLLGAATT (SEQ ID NO: 43).

The β-body TTVTGTLYPGTLLGFETT (SEQ ID NO: 41) was further modified to be in a cyclic form:

Abu(N₃)-TVTGTLYPGTLLGFET-Pra (SEQ ID NO: 44), and Pra-TVTGTLYPGTLLGFET-Abu(N₃) (SEQ ID NO: 34);

Its affinity for to the glycine receptor binding site of gephyrin (peptide binding site) was unchanged.

### Example 7

### Heterodimers

β-bodies can be designed to have a higher propension to form heterodimers than homodimers.

The following two β-bodies were designed:

(1)
(SEQ ID NO: 57)
TYTYTYPGLTRTHT
(2)
(SEQ ID NO: 58)
TTYTYPGDTFTI
(3)
(SEQ ID NO: 59)
TFTFTFPGLTRTHT
(4)
(SEQ ID NO: 60)
TDTRTYTYTVPGRTRTRTWTET,
and
(5)
(SEQ ID NO: 61)
DTITYTYTGPGRTDTETNTEG.

A fluorescent probe was attached to (1), (2) AND (3). The fluorescent β-bodies were then incubated with:
Beads modified with NHAc; or
Beads modified with (1); or
Beads modified with (2).

FIG. 8 shows that (1) and (2) could best bind beads modified with (2) and (1), respectively (heterodimers) (FIG. 8D and E). They could also bind with high affinity beads modified with (1) and (2), respectively (homodimers) (FIG. 8C and F). (3) is modified version of (2) were the tyrosine residues (Y) were substituted with phenylalanine residues. This substitution resulted in a 7× reduction of binding, from a K_{d} of 7*10⁻⁷ for bead-(2)—fluorescent-(1) to a K_{d} of 5*10⁻⁶ for bead-(2)—fluorescent-(3). The binding of the fluorescent 13-bodies to the unspecific NHAc-modified beads was minor (FIG. 8A and B).

### Example 8

β-bodies can also be used to neutralize toxins. To demonstrate this, β-bodies that bind toxin A from Clostridium difficile, the botulin toxin and ricin were designed according:
Clostridium difficile from 2G7CA: wEHTHTeTNPGNTYTST (SEQ ID NO: 29);
Botulinum toxin from the structure 4JRA.pdb: E-Abu(N₃)FTMEQTWTGPGSTKTFTFTH-Pra-G (SEQ ID NO: 30);
Ricin from the structure 4Z9K.pdb: LTFTFTVTPGTFTWTGTKPGETYTFTRTE (SEQ ID NO: 31).

The β-body targeting toxin A from Clostridium difficile binds the carbohydrate site where it substitutes the tetrasaccharide and cause neutralization of toxin A.

This example illustrates how β-bodies can be used for neutralizing toxins.

### Example 9

### β-Bodies can also be Used to Inhibit Proteases

Subtilisin and papain were chosen as test proteases for inhibition with both inwards (recognition) residues pointing towards the center and outward oriented β-bodies.

The following inwards oriented β-bodies were designed for subtilisin from the structure 1NDQ.pdb, D-amino acids:

tltmtwtythtpGtitwtytdtttG-OH; SEQ ID NO: 47; and

abu(N₃)-ItmtwtythtpGtitwtytdtt-pra-G-OH; SEQ ID NO: 48;

Figure YY shows the two sides of the β-body—active site interaction. The fit is extremely good at the bottom of the cleft.

The following inwards oriented β-bodies were designed for Papain from the structure 9PAP.pdb.

The inwards oriented β-body gives an extremely good fit to both plateaus in the binding site of papain and should interact strongly. All D-amino acid peptides:

abu(N₃)-qtmtGtItwtpGtqtntltwtf-pra-G (SEQ ID NO: 51); and tqtmtGtItwtpGtqtntltwtftG (SEQ ID NO: 50).

The following outwards oriented β-bodies were designed for papain, comprising D-amino acids (including 4-azido-2-aminobutanoic acid and propargylglycin) in addition to the inward oriented structures above:

abu(N₃)-wtftlpqGtmtn-pra-G; SEQ ID NO: 52, targeting plateau 1; and

abu(N₃)-wtvtvtfpGitmtttf-pra-OH; SEQ ID NO: 54, targeting plateau 2.

etltwtgtvtvtfpGitmtttEtmtftf-OH; SEQ ID NO: 53, this is a combination of the two peptides above but comprising a L-Glu, and targets both plateaus.

High concentration of β-body is used so that the proteolytic activity is balanced and inhibition is achieved. If concentration is not high enough the protease may cleave and deactivate the β-bodies.

### REFERENCES

Christensen, C., Bruun Schiodt, C., Taekker Foged, N., & Meldal, M. (2003). Solid Phase Combinatorial Library of 1,3-Azole Containing Peptides for the Discovery of Matrix Metallo Proteinase Inhibitors. QSAR & Combinatorial Science, 22(7), 754-766.

Lam, K. S.; Krchnak. V.; Lebl. M. Chem. Rev. 1997, 97, 411-448

Lam, K. S., Salmon, S. E., Hersh, E. M., Hruby, V., Kazmierski, W. M. and Knapp, R. J. ,A new type of synthetic peptide library for identifying ligand-binding activity, Nature, 354 (1991) 82-84.

Meldal, Morten, Christensen, Soren Flygering, 2010, Microparticle Matrix Encoding of Beads, Angewandte Chemie International Edition, Vol. 49, Issue 20, p.3473-3476.

1.15. (canceled)

16. A β-body, wherein the β-body is a compound comprising or consisting of at least two β-strand peptide sequences connected by β-turn peptide sequence(s), wherein said β-strand peptide sequences are organized in an anti-parallel arrangement of alternating forward and reverse β-strand peptide sequences, wherein each forward β-strand peptide sequence individually has the following sequence
Xᵣ(ZX)ₘ
and each reverse β-strand peptide sequence individually has the following sequence
(XZ)ₙXᵣ
wherein
each Z individually is Thr, a polar β-branched amino acid, non-proteinogenic α-branched amino acids that promote β-strand structure or a strand bridging amino acid, with the exception that at the most two Z in each β-strand sequence may be an amino acid, which is not one of the aforementioned,
and provided that at least 70% of the Z within each β-strand peptide sequences are Thr;
each X individually is any amino acid, β-amino acid or γ-amino acid; and
each m and n individually are integers in the range of 3 to 12; and
each r is an integer in the range of 0 to 5; and
and each β-turn peptide sequence individually has the following sequence
X_{q1}BUX_{q2}
wherein
each X individually is any amino acid;
each U individually is an amino acid of the formula 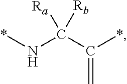
wherein Ra and Rb individually are selected from the group consisting of —H and C₁₋₆-alkyl, wherein Ra and Rb may be linked to form a cyclic structure;
B is selected from the group consisting of Pro, substituted Pro and pipecolic acid;
each q individually is an integer in the range of 0 to 5, wherein q1-q2 is −4, −2, 0, 2 or 4; and
wherein the β-body is linear or cyclic.

17. The β-body according to claim 1, wherein the compound comprises in the range of 2 to 10 β-strand peptide sequences connected by β-turn peptide sequences.

18. The β-body according to any one of the preceding claims, wherein said compound have the following structure:
forward β-strand sequence
β-turn peptide sequence
reverse β-strand sequence,
wherein the forward β-strand sequence and the reverse β-strand sequence are arranged as antiparallel β-strands.

19. The β-body according to any one of the preceding claims, where at least one forward β-strand sequence has the following sequence
Xᵣ(TX)ₘ
wherein
T is Thr;
each X individually is any amino acid; and
each m individually is an integer in the range of 3 to 12; and
r is an integer in the range of 0 to 5.

20. The β-body according to any one of the preceding claims, where at least one reverse β-strand sequence has the following sequence
(XT)ₙXᵣ
wherein
each X individually is any amino acid; and
each n individually is an integer in the range of 3 to 12; and
r is an integer in the range of 0 to 5.

21. The β-body according to any one of the preceding claims, wherein at least one β-turn peptide sequence has the following sequence
X_{q}PGX_{q}
wherein
each X individually is any amino acid, β-amino acid or γ-amino acid; and
each q individually is an integer in the range of 0 to 3.

22. The β-body according to any one of the preceding claims, wherein all of the amino acid residues of said β-body are L-amino acids.

23. The β-body according to any one of the preceding claims, wherein all of the amino acid residues of said β-body are D-amino acids.

24. The β-body according to any one of the preceding claims, wherein said compound is capable of binding a target compound with a K_{d} of at the most 10⁻⁶ M, for example 10⁻⁷ M or less, such as 10⁻⁸ M or less, such as 10⁻⁹ M or less, for example 10⁻¹⁰ M or less, or even 10⁻¹¹ M or even less.

25. The β-body according to any one of the preceding claims, wherein said β-body comprises or consists of an amino acid sequence selected from a group consisting of SEQ ID NO:1 to SEQ ID NO: 61.

26. A method for identifying a β-body according to any one of the preceding claims, wherein said β-body is capable of binding a target compound, said method comprising the steps of
a. Providing a spatial structure representation of the target compound in a computer;
b. Generating spatial structure representations of a plurality of β-bodies according to any one of the preceding claims in the computer;
c. selecting β-bodies fitting at least part of the spatial structure of the target compound in said computer
thereby identifying a β-body capable of binding the target compound.

27. A method for detecting the presence of a target compound in a sample, said method comprising
a. Providing a sample
b. Providing a β-body according to any one of claims 1 to 11, wherein said β-body is capable of binding said target compound
c. Incubating said sample with said β-body
d. Detecting β-bodies bound to said sample

28. A dimer comprising a first and a second β-body according to any one of claims 1 to 35, wherein said first β-body is different from the second β-body, or said first and said second β-body are identical and wherein said first and second β-bodies are capable of binding each other.

29. A compound comprising the β-body according to any one of claims 1 to 13, wherein said β-body is covalently linked to a conjugated moiety.

30. The β-body according to claim 1, where at least one forward β-strand sequence has the following sequence: Xᵣ(TX)ₘ,
and where at least one reverse β-strand sequence has the following sequence: (XT)ₙXᵣ
wherein T is Thr;
each X individually is any amino acid; and
each m individually is an integer in the range of 3 to 12;
each n individually is an integer in the range of 3 to 12; and
r is an integer in the range of 0 to 5;
wherein at least one β-turn peptide sequence has the following sequence: PG; and
wherein the β-body is linear or cyclic.
